SEQUENCE LISTING (57 sequences)

SEQ ID NO: 1 - protein, 245 aa, Arabidopsis thaliana
MGSKTEMMERDAMATVAPYAPVTYHRRARVDLDDRLPKPYMPRALQAPDREHPYGTPGHKNYGLSVLQQHVSFFDIDDNGIIYPWETYSGLRMLGFNIIGSLIIAAVINLTLSYATLPGWLPSPFFPIYIHNIHKSKHGSDSKTYDNEGRFMPVNLELIFSKYAKTLPDKLSLGELWEMTEGNRDAWDIFGWIAGKIEWGLLYLLARDEEGFLSKEAIRRCFDGSLFEYCAKIYAGISEDKTAYY

SEQ ID NO: 2 - protein, 244 aa, Arachis hypogaea [sequence omitted]

SEQ ID NO: 3 - protein, 245 aa, Sesamum indicum
MATHVLAAAAERNAALAPDAPLAPVTMERPVRTDLETSIPKPYMARGLVAPDMDHPNGTPGHVHDNLSVLQQHCAFFDQDDNGIIYPWETYSGLRQIGFNVIASLIMAIVINVALSYPTLPGWIPSPFFPIYLYNIHKAKHGSDSGTYDTEGRYLPMNFENLFSKHARTMPDRLTLGELWSMTEANREAFDIFGWIASKMEWTLLYILARDQDGFLSKEAIRRCYDGSLFEYCAKMQRGAEDKMK

SEQ ID NO: 4 - protein, 242 aa, Pinus massoniana
MASNESLQTTAAMAPVTIERRVNPNLDDELPKPFLPRALVAVDTEHPSGTPGHQHGDMSVLQQHVAFSNRNNDGIVYPWETFLGFRAVGFNIIISFFGCLIINIFLSYPTLPGWIPSPFFPIYIDRIHRAKHGSDSEVYDTEGRFVPAKFEEIFTKNAKTHPDKLSFSELWNLTEHNRNALDPLGWIAAKLEWFLLYSLAKDPHGFVPKEAARGVFDGSLFEFCEKSRKVKQATVKSLTFKI

SEQ ID NO: 5 - protein, 245 aa, Brassica napus
MSTATEIMERDAMATVAPYAPVTFHRRARVDMDDRLPKPYMPRALQAPDREHPYGTPGHKNYGLSVLQQHVAFFDLDDNGIIYPWETYSGLRMLGFNIIVSLIAAAVINLALSYATLTGWFPSPFFPIYIHNIHKSKHGSDSRTYDNEGRFMPVNLELIFSKYAKTLPDKLSLGELWEMTQGQRDAWDIFGWFASKIEWGLLYLLARDEEGFLSKEAIRRCFDGSLFEYCAKIYAGINEDKTAYY

SEQ ID NO: 6 - protein, 226 aa, Zea mays
MEVGRTPRRRASPAAAAAAAAAAVPSLLLFAVLFVGRAAAALGGPGPALYKHASFFDRDGDGVVSFAETYGAFRALGFGLGLSSASAAFINGALGSKCRPQNATSSKLDIYIEDIRRGKHGSDSGSYDAQGRFVPEKFEEIFARHARTVPDALTSDEIDQLLQANREPGDYSGWAGAEAEWKILYSLGKDGDGLLRKDVARSVYDGTLFHRLAPRWKSPDSDMERS

SEQ ID NO: 7 - DNA, 1044 bp, Arabidopsis thaliana
aaagtgagagagagatggggtcaaagacggagatgatggagagagacgcaatggctacggtggctccctatgcgccggtcacttaccatcgccgtgctcgtgttgacttggatgatagacttcctaaaccttatatgccaagagcattgcaagcaccagacagagaacacccgtacggaactccaggccataagaattacggacttagtgttcttcaacagcatgtctccttcttcgatatcgatgataatggcatcatttacccttgggagacctactctggactgcgaatgcttggtttcaatatcattgggtcgcttataatagccgctgttatcaacctgacccttagctatgccactcttccggggtggttaccttcacctttcttccctatatacatacacaacatacacaagtcaaagcatggaagtgattcaaaaacacatgacaatgaaggaaggtttatgccggtgaatcttgagttgatatttagcaaatatgcgaaaaccttgccagacaagttgagtcttggagaactatgggagatgacagaaggaaaccgtgacgcttgggacatttttggatggatcgcaggcaaaatagagtggggactgttgtacttgctagcaagggatgaagaagggtttttgtcaaaagaagctattaggcggtgtttcgatggaagcttgttcgagtactgtgccaaaatctacgctggtatcagtgaagacaagacagcatactactaaaagtatcctttatgttaagtaattgatcgagccattttaagctaataatcgatcaatgtgaagcttgtgcctatacggtaaatgaaggttcgggtagtagtatggacttttggtctaagagatctatgtttgtttttgtttttccagttctgtatggttatactataagttgcagctctaaagaaaagcttctgtatgttttgttgccttggtctctctttgtaccaacccctttttctgttatttccaattttacactgttagttattattgctgaaaaaaaaaaaaaaaa

SEQ ID NO: 8 - DNA, 1067 bp, Arachis hypogaea
gcaattttgcaaagcgagaaattccacacaggttacaccagtatatacgcatcttgctaaaccgactactgatcgagatcgctatggcggaggaggcggctagcaaggcagcgccgaccgatgcgctgtcgtccgtggcggcggaggcgccggtgacgagagaacggccggtccgagcggacttggaagtgcagattccgaagccctatttggcccgagctctggttgctccggacgtgtaccatcctgaaggaaccgaggggcgtgaccaccggcagatgagtgtgctgcagcagcatgtggctttcttcgacctggatggcgacggtatcgtttatccatgggaaacttatggaggactacgggaattgggcttcaacgtgattgtttcgttctttttggcgatagccataaacgttggtctaagctacccaactctgccaagctggataccatctctcctgttccctatacacataaaaaacatccacagggctaagcacggcagcgatagctcgacgtacgacaacgagggaaggtttatgccggtcaatttcgagagcatcttcagcaagaacgcccgcacggcgccggacaagctcacgttcggcgatatctggcggatgaccgaaggccaaagggtggcgctcgacttgcttgggaggatcgcgagtaagggggagtggatattgctctacgtgcttgcgaaagatgaggaaggattcctcaggaaggaggctgttcgccgctgcttcgatgggagcctattcgagtcgattgcccagcagagaagggaggcacatgagaagcagaagtagcctcctaatttcatcgtcccgggacctgggatgtgcttgattgcttgtgtgtgttgttgtgtggactatagctatagccacatcatgtttgtccatctgaaaaaacaatggaaataaggtttaccggttggaacatacattatgtactatccatgtgattattgaaatgtgtctgtaacctgaaagtgtgattgacatataaaattctgtgattgaagtaaaggtaagcattaaaaaaaaaaaaaaaaa

SEQ ID NO: 9 - DNA, 1030 bp, Sesamum indicum
ggcacgagagagaaaaaaggtgattttgtcaagggaaatatggcaactcatgttttggctgctgcggcggagagaaatgctgcgttggcgccggacgccccgcttgctccggtgactatggagcgcccagtgcgcactgacttggagacttcgatcccgaagccctatatggcaagaggattggttgcacctgatatggatcaccccaacggaacaccaggccatgtgcatgataatttgagtgtgctgcaacagcattgtgctttctttgatcaggatgataacggaatcatctatccatgggagacttactctggacttcgccaaattggtttcaatgtgatagcttcccttataatggctatcgtcattaatgtggcgctgagttatcctactctcccgggttggattccttctccttttttccccatatatttgtacaacatacacaaggccaaacatggaagcgactccggaacctatgatactgaaggaaggtacctacctatgaattttgagaacctgttcagcaagcatgcccggacaatgcccgataggctcactctaggggagctatggagcatgactgaagctaacagagaagcatttgacattttcggctggatcgcaagcaaaatggagtggactctcctctacattcttgcaagagaccaggacggtttcctgtcgaaagaagccatcaggcggtgttacgatggcagtttgttcgagtactgtgcaaagatgcaaaggggagccgaggacaagatgaaatgaaggaaatcggctatcgcggtaggtgtaagttatgatgtggtgtgtatgatggattgaaagtgccagtgcttaagttgtgtggcagagtcttgtgtaataacctttgtgtacagatttaaggtctcggaattggtgtaactgtggagaagatgttgactcctgtttttgttcaataagtccaactcttgacatttggttggtttgcagggaaagatggggaattttgttttccgaaaaaaaaaaaaaaaaaaaa

SEQ ID NO: 10 - DNA, 1178 bp, Pinus massoniana
atgggggtgctgcaaaaaaaattgaacttcatcaaatctagttccaggaattgtaggtcgcgaggtcggatctgtgggactgagcaaattattatcactgtgatcgagaaagcatttaagtaccagctataatggcttccaatgaatctttacagacaacagctgctatggcaccagtaacaatcgagcgcagggttaaccccaatctcgatgacgaactcccaaaaccttttctcccaagggcgctcgtagcagttgacacagaacatccgagtggaacccctggacaccaacacggcgacatgagcgttcttcaacagcacgtcgcattttccaatcgcaacaacgacgggattgtgtacccttgggagactttcttaggttttcgtgccgtgggttttaatataataatctcgttctttggttgccttattatcaacattttcttgagctatcctacgttgcctggatggattccctcgccattttttccaatctatattgataggattcatcgagcgaagcatggaagcgattccgaagtttatgacacagaaggaaggtttgtccccgctaaattcgaagaaatttttacaaaaaatgccaaaacccatccagataaactgtcattctctgagctgtggaatttgacggaacacaatagaaatgcgcttgatcctttaggatggattgcggcgaagttagaatggttcttgttatactctctggctaaagacccccatggttttgtgcccaaggaagctgcgagaggtgtatttgatggtagcttgttcgagttctgcgagaagtctcgaaaggtcaaacaagcaacagtgaaatccctgacctttaagatttgaagctctaaaaactcttgcggtcattgtcataaattggtgctctctttatgtctataaggtggactactctacaagatgggctgccatgtatatataggaagatatgcattgaagtaggaatcaactggttgagcctcttctagatggaagattgtagagtcatgaaacctccctcccatataagtaagacaatattagtcagaagagagaaaaatctctgcgtgataccactgctgcctaaagaagtcgattagaatcactagtgatcgcgccgctgcagtcgaacatatgggaagctcccaccgtgatgcaagctga

SEQ ID NO: 11 - DNA, 1030 bp, Brassica napus
tacggccggggattgcactcggtccacagagcaagaaagagcgagagatgagtacggcgactgagataatggagagagacgcaatggctacggtggctccctacgctccggtcacctttcaccgccgtgctcgtgttgacatggatgatagacttcctaaaccttatatgccaagagcactgcaagcacccgacagagagcatccgtatggaaccccaggccataagaattatggacttagtgttcttcagcaacatgtcgccttcttcgatttagatgataatggaattatctatccttgggagacctactctggactgcgaatgctaggtttcaatatcattgtatcgcttatcgcagccgctgtaatcaacttggcccttagctatgctactcttacgggatggtttccttcgccgttcttcccaatatacatacacaatatacacaagtcaaagcatgggagcgactcaagaacatatgacaatgaagggaggtttatgcctgtgaatcttgagttgatatttagcaaatatgcgaaaacattgccagacaagttgagtcttggagaattatgggagatgacacaaggacaacgtgacgcatgggacatcttcggatggttcgcaagcaaaatagagtgggggttgttgtacttgctagcgagggatgaagaagggtttctgtcaaaagaagcgattaggaggtgttttgacgggagcttgttcgagtattgtgccaagatctacgcaggtatcaatgaagacaagacagcctactactaaaagtaaatgatagaggagctttaggctgataatcgtccatgtgaatgtaacttgtgtctaaagcagagtccatgtgtttgttatgttatgtccaaatctgtaaggtagagtatcatcagttgcagctggtatagaaagcttctatgatcataatatagtatgtttgtgtgggttgtgttgggttgatcacccttttcagtattcaggtcaatgtattttcatggtgtagaggaaaaaaaaaaaaa

SEQ ID NO: 12 - DNA, 1012 bp, Zea mays
aagctgcgctgccagtgccagcgctcactcgaacgccgagacccgagaggagcaaacagccaaaaagaacggaaaggggagagcaaacagccaaaaaaggacggacttgcgcgacagggtcgaagactcagaaggggaatctccggaggatggaggtgggcaggactccgcggcgacgggcgtccccagcggcagcggcggcggcggcggcggcggctgtgccttcgctgcttctgttcgccgtgctattcgtgggccgggcggcggcagcgttgggcggcccggggccggcgctatacaagcacgcgtcgttcttcgaccgcgacggcgacggcgtcgtctccttcgcggagacgtacggcgcgtttcgggccctcgggtttggactcggcctgtccagcgccagcgccgccttcatcaatggcgcccttggcagcaagtgcagacctcaaaacgcgacgtcgtcgaaactggacatctacatagaggacatccggagagggaagcacgggagcgactccggctcgtacgacgcccaaggaaggttcgttccggagaagttcgaggagatattcgccaggcacgcgaggacggtccccgacgccctgacctcggacgagatcgaccagctgctccaagcgaacagagagcccggggactacagcggctgggctggcgcggaagcggagtggaagatcctgtacagtctcggcaaggacggggacggcctcctccgcaaggacgtcgcgaggagcgtctacgacgggacactgttccaccggctcgcgcccagatggaaatctcccgacagcgacatggagagaagctgataagcgtggtccgggagaactgaaccgagaggaccgtcctattgatgtcgtcttgcgctgggctgctctgaactgaacaagtctggacatgccgtcaagcgacatgtgggtgtgaacactctttcgggtcagattattaacaagaagggtgtgaccgtgtgagtgcaaaaaaaaaaaaaaaa

SEQ ID NO: 13 - protein, 92 aa, Arabidopsis thaliana
MAGEAEALATTAPLAPVTSQRKVRNDLEETLPKPYMARALAAPDTEHPNGTEGHDSKGMSVMQQHVAFFDQNDDGIVYPWETYKGFRDLGFN

SEQ ID NO: 14 - protein, 100 aa, Sesamum indicum
MATHVLAAAAERNAALAPDAPLAPVTMERPVRTDLETSIPKPYMARGLVAPDMDHPNGTPGHVHDNLSVLQQHCAFFDQDDNGIIYPWETYSGLRQIGFN

SEQ ID NO: 15 - protein, 99 aa, Oryza sativa
MAEEAASKAAPTDALSSVAAEAPVTRERPVRADLEVQIPKPYLARALVAPDVYHPEGTEGRDHRQMSVLQQHVAFFDLDGDGIVYPWETYGGLRELGFN

SEQ ID NO: 16 - protein, 94 aa, Artificial Sequence
  Synthetic Construct
MAAEMERESLITEAPNAPVTAQRRVRNDLENSLPKPYLPRALKAPDTGHPNGTAGHRHHNLSVLQQHCAFFDQDDNGIIYPWETYMGLRSIGFN

SEQ ID NO: 17 - DNA, 276 bp, Arabidopsis thaliana
atggcaggagaggcagaggctttggccacgacggcaccgttagctccggtcaccagtcagcgaaaagtacggaacgatttggaggaaacattaccaaaaccatacatggcaagagcattagcagctccagatacagagcatccgaatggaacagaaggtcacgatagcaaaggaatgagtgttatgcaacaacatgttgctttcttcgaccaaaacgacgatggaatcgtctatccttgggagacttataagggatttcgtgaccttggtttcaac

SEQ ID NO: 18 - DNA, 300 bp, Sesamum indicum
atggcaactcatgttttggctgctgcggcggagagaaatgctgcgttggcgccggacgccccgcttgctccggtgactatggagcgcccagtgcgcactgacttggagacttcgatcccgaagccctatatggcaagaggattggttgcacctgatatggatcaccccaacggaacaccaggccatgtgcatgataatttgagtgtgctgcaacagcattgtgctttctttgatcaggatgataacggaatcatctatccatgggagacttactctggacttcgccaaattggtttcaat

SEQ ID NO: 19 - DNA, 297 bp, Oryza sativa
atggcggaggaggcggctagcaaggcagcgccgaccgatgcgctgtcgtccgtggcggcggaggcgccggtgacgagagaacggccggtccgagcggacttggaagtgcagattccgaagccctatttggcccgagctctggttgctccggacgtgtaccatcctgaaggaaccgaggggcgtgaccaccggcagatgagtgtgctgcagcagcatgtggctttcttcgacctggatggcgacggtatcgtttatccatgggaaacttatggaggactacgggaattgggcttcaac

SEQ ID NO: 20 - DNA, 282 bp, Artificial Sequence
  Synthetic Construct
atggctgcagagatggagagggagtcattgataactgaagctcctaatgcaccagttactgcacagagaagggtcagaaatgacttagaaaattctctaccaaaaccatacttgccaagagcattgaaagctcctgatacgggtcacccaaatggaacagcaggccacaggcaccacaacttatctgttcttcagcagcattgtgctttttttgatcaagatgacaatggaatcatttacccttgggaaacttacatggggctgcgttctattggatttaat

SEQ ID NO: 21 - protein, 36 aa, Arabidopsis thaliana
PISSIFWTLLINLAFSYVTLPSWVPSPLLPVYIDNI

SEQ ID NO: 22 - protein, 36 aa, Sesamum indicum
VIASLIMAIVINVALSYPTLPGWIPSPFFPIYLYNI

SEQ ID NO: 23 - protein, 36 aa, Oryza sativa
VIVSFFLAIAINVGLSYPTLPSWIPSLLFPIHIKNI

SEQ ID NO: 24 - protein, 36 aa, Artificial Sequence
  Synthetic Construct
VVASVIMAIVINVGLSYPTLPNWFPSLLFPIYIHNI

SEQ ID NO: 25 - DNA, 108 bp, Arabidopsis thaliana
ccaatttcctctatcttttggaccttactcataaacttagcgttcagctacgttacacttccgagttgggtgccatcaccattattgccggtttatatcgacaacata

SEQ ID NO: 26 - DNA, 108 bp, Sesamum indicum
gtgatagcttcccttataatggctatcgtcattaatgtggcgctgagttatcctactctcccgggttggattccttctccttttttccccatatatttgtacaacata

SEQ ID NO: 27 - DNA, 108 bp, Oryza sativa
gtgattgtttcgttctttttggcgatagccataaacgttggtctaagctacccaactctgccaagctggataccatctctcctgttccctatacacataaaaaacatc

SEQ ID NO: 28 - DNA, 108 bp, Artificial Sequence
  Synthetic Construct
gttgttgcatctgttattatggctattgttatcaatgttggattgagttaccccactctacctaattggttcccttctctcctttttcctatctacatacacaacata

SEQ ID NO: 29 - protein, 66 aa, Arabidopsis thaliana
HKAKHGSDSSTYDTEGRLSNKVEWILLYILAKDEDGFLSKEAVRGCFDGSLFEQIAKERANSRKQD

SEQ ID NO: 30 - protein, 109 aa, Sesamum indicum
HKAKHGSDSGTYDTEGRYLPMNFENLFSKHARTMPDRLTLGELWSMTEANREAFDIFGWIASKMEWTLLYILARDQDGFLSKEAIRRCYDGSLFEYCAKMQRGAEDKMK

SEQ ID NO: 31 - protein, 109 aa, Oryza sativa
HRAKHGSDSSTYDNEGRFMPVNFESIFSKNARTAPDKLTFGDIWRMTEGQRVALDLLGRIASKGEWILLYVLAKDEEGFLRKEAVRRCFDGSLFESIAQQRREAHEKQK

SEQ ID NO: 32 - protein, 109 aa, Artificial Sequence
  Synthetic Construct
HKAKHGSDSGVYDTEGRYVPANIENIFSKYARTVPDKLTLGELWDLTEGNRNAFDIFGWLAAKFEWGVLYILARDEEGFLSKEAVRRCFDGSLFEYCAKMHTTSDAKMS

SEQ ID NO: 33 - DNA, 198 bp, Arabidopsis thaliana
cacaaagccaagcatgggagtgattcgagcacctatgacaccgaaggaaggctttcaaacaaagttgaatggatactactctatattcttgctaaggacgaagatggtttcctatctaaagaagctgtgagaggttgctttgatggaagtttatttgaacaaattgccaaagagagggccaattctcgcaaacaagac

SEQ ID NO: 34 - DNA, 327 bp, Sesamum indicum
cacaaggccaaacatggaagcgactccggaacctatgatactgaaggaaggtacctacctatgaattttgagaacctgttcagcaagcatgcccggacaatgcccgataggctcactctaggggagctatggagcatgactgaagctaacagagaagcatttgacattttcggctggatcgcaagcaaaatggagtggactctcctctacattcttgcaagagaccaggacggtttcctgtcgaaagaagccatcaggcggtgttacgatggcagtttgttcgagtactgtgcaaagatgcaaaggggagccgaggacaagatgaaa

SEQ ID NO: 35 - DNA, 327 bp, Oryza sativa
cacagggctaagcacggcagcgatagctcgacgtacgacaacgagggaaggtttatgccggtcaatttcgagagcatcttcagcaagaacgcccgcacggcgccggacaagctcacgttcggcgatatctggcggatgaccgaaggccaaagggtggcgctcgacttgcttgggaggatcgcgagtaagggggagtggatattgctctacgtgcttgcgaaagatgaggaaggattcctcaggaaggaggctgttcgccgctgcttcgatgggagcctattcgagtcgattgcccagcagagaagggaggcacatgagaagcagaag

SEQ ID NO: 36 - DNA, 327 bp, Artificial Sequence
  Synthetic Construct
cacaaagcaaagcatgggagtgactctggagtttatgacacagaaggacgttatgtgccagcaaatattgagaacatattcagtaagtatgctcgtacagtacctgacaagctcacacttggggagctctgggacttgacagagggaaaccgaaatgcttttgacatatttggctggcttgcagcaaaatttgaatggggggttctgtacattctggcaagggatgaggaaggtttcctgtctaaagaagctgttagaagatgctttgatgggagcttatttgaatactgtgctaaaatgcatactactagtgatgccaagatgagt

SEQ ID NO: 37 - protein, 7 aa, Artificial Sequence
  Synthetic Construct
ENLYFQS

SEQ ID NO: 38 - DNA, 21 bp, Artificial Sequence
  Synthetic Construct
gagaacctctacttccaatcg

SEQ ID NO: 39 - protein, 198 aa, Artificial Sequence
  Synthetic Construct
CITGDALVALPEGESVRIADIVPGARPNSDNAIDLKVLDRHGNPVLADRLFHSGEHPVYTVRTVEGLRVTGTANHPLLCLVDVAGVPTLLWKLIDEIKPGDYAVIQRSAFSVDCAGFARGKPEFAPTTYTVGVPGLVRFLEAHHRDPDAQAIADELTDGRFYYAKVASVTDAGVQPVYSLRVDTADHAFITNGFVSHA

SEQ ID NO: 40 - DNA, 594 bp, Artificial Sequence
  Synthetic Construct
tgcatcacgggagatgcactagttgccctacccgagggcgagtcggtacgcatcgccgacatcgtgccgggtgcgcggcccaacagtgacaacgccatcgacctgaaagtccttgaccggcatggcaatcccgtgctcgccgaccggctgttccactccggcgagcatccggtgtacacggtgcgtacggtcgaaggtctgcgtgtgacgggcaccgcgaaccacccgttgttgtgtttggtcgacgtcgccggggtgccgaccctgctgtggaagctgatcgacgaaatcaagccgggcgattacgcggtgattcaacgcagcgcattcagcgtcgactgtgcaggttttgcccgcgggaaacccgaatttgcgcccacaacctacacagtcggcgtccctggactggtgcgtttcttggaagcacaccaccgagacccggacgcccaagctatcgccgacgagctgaccgacgggcggttctactacgcgaaagtcgccagtgtcaccgacgccggcgtgcagccggtgtatagccttcgtgtcgacacggcagaccacgcgtttatcacgaacgggttcgtcagccacgct

SEQ ID NO: 41 - DNA, 496 bp, Artificial Sequence
  Synthetic Construct
tcgctgaggcttgacatgattggtgcgtatgtttgtatgaagctacaggactgatttggcgggctatgagggcgggggaagctctggaagggccgcgatggggcgcgcggcgtccagaaggcgccatacggcccgctggcggcacccatccggtataaaagcccgcgaccccgaacggtgacctccactttcagcgacaaacgagcacttatacatacgcgactattctgccgctatacataaccactcagctagcttaagatcccatcaagcttgcatgccgggcgcgccagaaggagcgcagccaaaccaggatgatgtttgatggggtatttgagcacttgcaacccttatccggaagccccctggcccacaaaggctaggcgccaatgcaagcagttcgcatgcagcccctggagcggtgccctcctgataaaccggccagggggcctatgttctttacttttttacaagagaagtcactcaacatcttaaa

SEQ ID NO: 42 - protein, 10 aa, Arabidopsis thaliana
PSWVPSPLLP

SEQ ID NO: 43 - protein, 10 aa, Sesamum indicum
PGWIPSPFFP

SEQ ID NO: 44 - protein, 10 aa, Oryza sativa
PSWIPSLLFP

SEQ ID NO: 45 - protein, 10 aa, Artificial Sequence
  Synthetic Construct
PNWFPSLLFP

SEQ ID NO: 46 - DNA, 30 bp, Arabidopsis thaliana
ccgagttgggtgccatcaccattattgccg

SEQ ID NO: 47 - DNA, 48 bp, Sesamum indicum
ccgggttggattccttctccttttttccccatatatttgtacaacata

SEQ ID NO: 48 - DNA, 30 bp, Oryza sativa
ccaagctggataccatctctcctgttccct

SEQ ID NO: 49 - DNA, 30 bp, Artificial Sequence
  Synthetic Construct
cctaattggttcccttctctcctttttcct

SEQ ID NO: 50 - protein, 28 aa, Arabidopsis thaliana
MQQHVAFFDQNDDGIVYPWETYKGFRDL

SEQ ID NO: 51 - protein, 28 aa, Sesamum indicum
LQQHCAFFDQDDNGIIYPWETYSGLRQI

SEQ ID NO: 52 - protein, 50 aa, Oryza sativa
PDVYHPEGTEGRDHRQMSVLQQHVAFFDLDGDGIVYPWETYGGLRELGFN

SEQ ID NO: 53 - protein, 50 aa, Artificial Sequence
  Synthetic Construct
PDTGHPNGTAGHRHHNLSVLQQHCAFFDQDDNGIIYPWETYMGLRSIGFN

SEQ ID NO: 54 - DNA, 84 bp, Arabidopsis thaliana
atgcaacaacatgttgctttcttcgaccaaaacgacgatggaatcgtctatccttgggagacttataagggatttcgtgacctt

SEQ ID NO: 55 - DNA, 84 bp, Sesamum indicum
ctgcaacagcattgtgctttctttgatcaggatgataacggaatcatctatccatgggagacttactctggacttcgccaaatt

SEQ ID NO: 56 - DNA, 150 bp, Oryza sativa
ccggacgtgtaccatcctgaaggaaccgaggggcgtgaccaccggcagatgagtgtgctgcagcagcatgtggctttcttcgacctggatggcgacggtatcgtttatccatgggaaacttatggaggactacgggaattgggcttcaac

SEQ ID NO: 57 - DNA, 150 bp, Artificial Sequence
  Synthetic Construct
cctgatacgggtcacccaaatggaacagcaggccacaggcaccacaacttatctgttcttcagcagcattgtgctttttttgatcaagatgacaatggaatcatttacccttgggaaacttacatggggctgcgttctattggatttaat

(19) United States
(12) Patent Application Publication
(10) Pub. No.: US 2020/0172948 A1
(43) Pub. Date: Jun. 4, 2020

(54) RECOMBINANT POLYPEPTIDE ENRICHED ALGAL CHLOROPLASTS, METHODS FOR PRODUCING THE SAME AND USES THEREOF

(71) Applicant: Alcantara Research Group Inc., Calgary (CA)

(72) Inventor: Joenel ALCANTARA, Calgary (CA)

(21) Appl. No.: 16/631,501

(22) PCT Filed: Jul. 9, 2018
(86) PCT No.: PCT/CA2018/050831

§ 371 Date: Jan. 16, 2020

Related U.S. Application Data
(60) Provisional application No. 62/534,405, filed on Jul. 19, 2017.

(51) Int. Cl. C12P 21/02; C12N 15/82; C12N 1/12

(52) U.S. Cl. CPC C12P 21/02; C12N 1/12; C12N 15/8201

(57) ABSTRACT

The present invention relates to recombinant protein expression in algal cells. In particular, the present invention provides methods for making recombinant polypeptides in association with such chloroplast-associated oil body proteins such as caleosin or fragments thereof. In certain embodiments, the methods involve transformation of algal cells with a nucleic acid encoding a fusion protein comprising an oil body protein and a protein of interest and subsequently growing the algal cells under non-homeostatic conditions to induce accumulation of the fusion polypeptide in the chloroplast of said algal cells.

### FIELD OF THE INVENTION

This invention pertains to the field of recombinant polypeptide production in algae and in particular the production of recombinant polypeptide enriched algal chloroplasts.

### BACKGROUND OF THE INVENTION

A wide variety of techniques for the production of recombinant polypeptides in hosts are known in the art. Well-known examples of recombinant production hosts include cell culture-based host cell systems, such as microbial cell systems that use bacterial cells, fungal cells, yeast cells, as well as animal cell systems including mammalian and insect cell culture systems. Other techniques involve the generation of genetically modified plants and animals.

The benefits of using microbial cells for the production of recombinant polypeptides include the low costs associated with cultivation of microbial cells, substantial product yields, and limited toxicity of raw materials. On a larger scale, however, capital costs may become prohibitively expensive due to factors such as increased material requirements including growth media, scale-up of production facilities, and the expense associated with protein purification, notably in manufacturing operations designed to provide highly purified protein preparations, such as biopharmaceutical proteins.

Historically plants have represented an effective and economical method to produce recombinant polypeptides as they can be grown at a large scale with modest cost inputs. The use of plants has distinct advantages over bacterial systems as bacterial systems are frequently not appropriate for the production of many proteins due to differences in protein processing and codon usage. Although foreign proteins have successfully been expressed in plants, the development of systems that can offer commercially viable levels of expression and effective cost separation techniques are still needed. One of the methods which has been explored is the method of producing recombinant polypeptides in association with plant oil-bodies as documented in for example U.S. Pat. No. 5,650,554.

Eukaryotic microalgae, hereinafter “algae” or “algal cells”, are eukaryotic photosynthetic organisms that can readily be grown in a variety of environments, such as large-scale bioreactors, making them attractive candidates for recombinant polypeptide expression.

Techniques to introduce genes capable of expressing recombinant polypeptides in algal cells are well known in the art and research efforts have been made to utilize algae for the purposes of the production of biomolecules as detailed in U.S. Pat. Nos. 8,951,777; 9,315,837; United States Patent Application No 2011/0030097; United States Patent Application No. 2012/0156717; U.S. Pat. No. 6,157,517 and PCT Patent Application No WO2012047970.

Algae in principle represent an attractive eukaryotic cellular host system for the synthesis of polypeptides due to the relative ease with which algal cells may be grown, as well as the availability of genetic engineering techniques. In many instances, upon production of the recombinant polypeptide, it is desirable to separate the polypeptide of interest from algal cellular constituents. Known techniques for the isolation of proteins from algal cells include the performance of a wide variety of protein purification techniques, such as chromatographical techniques, including ion exchange chromatography, high performance liquid chromatography, hydrophobic interaction chromatography, and the like. While these techniques are suitable to obtain substantially pure protein preparations on a laboratory scale, they are often inherently impractical to implement on the commercial scale. Moreover, commercial scale protein purification techniques are often the most expensive operational step. Due to the paucity of efficient protein production and extraction techniques known to the art, the commercial manufacture of proteins using algal cells remains substantially economically unviable.

Accordingly, there exists a need for improved techniques for the production of recombinant polypeptides in algae that are readily adaptable to commercial scale operations.

This background information is provided for the purpose of making known information believed by the applicant to be of possible relevance to the present invention. No admission is necessarily intended, nor should be construed, that any of the preceding information constitutes prior art against the present invention.

### SUMMARY OF THE INVENTION

An object of the present invention is to provide recombinant polypeptide enriched algal chloroplasts, methods for producing the same and uses thereof. In accordance with an aspect of the invention, there is provided a method of producing an algal chloroplast enriched for recombinant polypeptide, the method comprising subjecting growing algal cells comprising a recombinant polypeptide to non-homeostatic conditions to target the recombinant polypeptide to the algal chloroplast; wherein the recombinant polypeptide is a fusion polypeptide comprising an oil body protein or fragment thereof. In some embodiments, the method comprising isolating the recombinant polypeptide enriched algal chloroplasts.

In accordance with another aspect of the invention, there is provided a method of producing algal chloroplasts enriched for recombinant polypeptide, the method comprising (a) introducing a nucleic acid into algal cells, the nucleic acid comprising as operably linked components (i) a nucleic acid encoding a fusion polypeptide comprising an oil body protein or fragment thereof to provide targeting to the algal chloroplast and a polypeptide of interest; and (ii) a nucleic acid sequence capable of controlling expression in an algal cell; (b) subjecting the algal cells in a growth medium to non-homeostatic conditions to target the fusion polypeptide to the algal chloroplast; and (c) optionally, isolating the algal chloroplasts.

In accordance with another aspect of the invention, there is provided a chloroplast isolated by the above method.

In accordance with some embodiments of the invention, the recombinant protein is isolated from the isolated chloroplasts.

In accordance with another aspect of the invention, there is provided an algal cell comprising fusion polypeptide comprising an oil body protein or fragment thereof and a protein of interest, wherein the oil body protein or fragment thereof targets the fusion polypeptide to chloroplasts when the cell is subjected to non-homeostatic conditions.

In accordance with another aspect of the invention, there is provided an algal cell comprising nucleic acid comprising as operably linked components (i) a nucleic acid sequence encoding fusion polypeptide comprising an oil body protein or fragment thereof and a protein of interest, wherein the oil body protein or fragment thereof targets the fusion polypeptide to chloroplasts when the cell is subjected to non-homeostatic conditions; and (ii) a nucleic acid sequence capable of controlling expression in an algal cell.

In accordance with another aspect of the invention, there is provided a preparation comprising chloroplasts wherein the chloroplasts comprise a fusion polypeptide comprising an oil body protein or fragment thereof and a protein of interest, wherein the oil body protein or fragment thereof targets the fusion polypeptide to chloroplasts when an algal cell is subjected to non-homeostatic conditions.

In accordance with another embodiment of the invention, there is provided a nucleic acid encoding a fusion polypeptide comprising an oil body protein or fragment thereof to provide targeting to algal chloroplast and a polypeptide of interest.

In accordance with some embodiments, the oil body protein is a caleosin, optionally encoded by a nucleic acid sequence having the sequence set forth in any one of SEQ.ID NO: 7 to SEQ.ID NO: 12.

In accordance with another aspect of the invention, there is provided a recombinant expression vector having a nucleic acid sequence encoding a fusion polypeptide comprising an oil body protein or fragment thereof to provide targeting to algal chloroplast and a polypeptide of interest operatively linked to a nucleic acid sequence capable of controlling expression in an algal cell; wherein the expression vector is suitable for expression in an algal cell.

In accordance with another aspect of the invention, there is provided a method of producing algae enriched for recombinant polypeptide, the method comprising subjecting growing algal cells comprising a recombinant polypeptide at over 22° C. and CO₂ over 0.5%; wherein the recombinant polypeptide is a fusion polypeptide comprising an oil body protein or fragment thereof and wherein optionally the algae clump together and the algae is isolated by removing the clumps.

In accordance with another aspect of the invention, there is provided a method of producing of producing a recombinant protein, the method comprising subjecting growing algal cells comprising a recombinant polypeptide at over 22° C. and CO₂ over 0.5%; wherein the recombinant polypeptide is a fusion polypeptide comprising an oil body protein or fragment thereof; allowing the algae clump together; isolating the algae by removing the clumps and isolating the recombinant polypeptide from the clumps.

### BRIEF DESCRIPTION OF THE DRAWINGS

Embodiments of the invention will now be described, by way of example only, by reference to the attached FIGURE, wherein:

FIG. 1 illustrates confocal microscopic images showing wild type algal cells and algal cells transformed with a plasmid that encodes a YFP recombinantly fused to a caleosin protein. Upon transformation and expression of the fusion polypeptide, the fusion polypeptide accumulates in the cytoplasm similar to other recombinant polypeptides. However, once the cells are subjected to nitrogen stress (removal of nitrogen from the growth media), the caleosin-YFP fusion is targeted to the chloroplast (autofluorescence).

### DETAILED DESCRIPTION OF THE INVENTION

### Definitions

Unless defined otherwise, all technical and scientific terms used herein have the same meaning as commonly understood by one of ordinary skill in the art to which this invention belongs.

The herein interchangeably used terms “nucleic acid sequence encoding a caleosin”, nucleic acid sequence encoding a caleosin protein” and “nucleic acid sequence encoding a caleosin polypeptide”, refer to any and all nucleic acid sequences encoding a caleosin, including but not limited to those set forth in SEQ.ID NO: 7 to SEQ.ID NO: 12 (see table). Nucleic acid sequences encoding a caleosin further include any and all nucleic acid sequences which (i) encode polypeptides that are substantially identical to the caleosin sequences set forth herein; or (ii) the complement of which hybridizes to any caleosin nucleic acid sequences set forth herein under at least moderately stringent hybridization conditions or which would hybridize thereto under at least moderately stringent conditions but for the use of synonymous codons.

The term “nucleic acid sequence encoding a central domain”, refers to any and all nucleic acid sequences encoding a central domain, including but not limited to the nucleic acid sequences set forth in SEQ.ID NO: 25 to SEQ.ID NO: 28 (see table). Nucleic acid sequences encoding a central domain further include any and all nucleic acid sequences which (i) encode polypeptides that are substantially identical to the central domain sequences set forth herein; or (ii) the complement of which hybridizes to any central domain nucleic acid sequences set forth herein under at least moderately stringent hybridization conditions or which would hybridize thereto under at least moderately stringent conditions but for the use of synonymous codons.

The term “nucleic acid sequence encoding a proline knot motif”, refers to any and all nucleic acid sequences encoding a proline knot motif, including but not limited to the nucleic acid sequence set forth in SEQ.ID NO: 46 to SEQ.ID NO: 49 (see table). Nucleic acid sequences encoding a proline knot motif further include any and all nucleic acid sequences which (i) encode polypeptides that are substantially identical to the proline knot motif sequences set forth herein; or (ii) the complement of which hybridizes to any proline knot motif nucleic acid sequences set forth herein under at least moderately stringent hybridization conditions or which would hybridize thereto under at least moderately stringent conditions but for the use of synonymous codons.

The term “homeostatic growth conditions” as used herein, in relation to the cultivation of algal cells, refers to growth conditions under which an algal cell culture is grown under substantially optimal growth conditions. Under homeostatic growth conditions algal cells in a cell culture may temporally exist in different growth phases, including a lag phase; a logarithmic growth phase, also known as exponential growth phase; a stationary phase; or a death phase. During each growth phase algal cells have a characteristic growth rate corresponding with such growth phase when grown under homeostatic growth conditions. Notably, under homeostatic growth conditions, during logarithmic growth phase, the algal cell population doubles at a constant rate. The rate with which a cell population doubles in size is also known and referred herein as the doubling rate.

The terms “non-homeostatic growth conditions” and “non-homeostatic conditions”, as used herein in relation to the cultivation of algal cells, refers to conditions and growth conditions substantially deviating from homeostatic growth conditions. Under non-homeostatic growth conditions the algal growth rate substantially deviates from the corresponding growth rate under homeostatic growth conditions. When the conditions are altered during the logarithmic phase from homeostatic growth conditions to non-homeostatic conditions the doubling rate decreases to a doubling rate that is lower than the doubling rate during the logarithmic phase under homeostatic growth conditions.

Overview:

The present invention provides a method of producing algal chloroplasts enriched for recombinant polypeptide. The method includes subjecting growing algal cells that express a fusion polypeptide to non-homeostatic conditions to target the recombinant polypeptide to the algal chloroplast. The fusion polypeptide includes an oil body protein or fragment thereof that targets the fusion polypeptide to the chloroplasts following stress or non-homeostatic conditions. In some embodiments, the fusion polypeptide includes caleosin or a targeting fragment thereof.

The recombinant polypeptide enriched chloroplasts may be isolated from the algae by various techniques known in the art. Optionally, the recombinant polypeptides can be isolated from the chloroplasts. Techniques for isolating the polypeptides from the chloroplasts are also known in the art

Alternatively, the recombinant polypeptide enriched chloroplasts are isolated for use in nutraceutical, pharmaceutical or other applications known in the art.

In some embodiments, the recombinant protein enriched chloroplasts or algae are used as a nutraceutical, optionally as a protein supplement.

In some embodiments, the invention provides a method of producing algae enriched for recombinant polypeptide by promoting the clumping of the algal cells. The method includes growing algal cells that express a fusion polypeptide to 22° C. and CO₂ over 0.5%. The fusion polypeptide includes an oil body protein or fragment thereof.

As hereinbefore mentioned, the present invention relates to processes for the manufacture of recombinant polypeptides in algal cells and in particular, in chloroplast contained therein.

In some embodiments, by targeting polypeptides to the chloroplast, recombinant polypeptide purification is facilitated as the recombinant polypeptide containing chloroplasts can readily be separated from other cellular constituents by methods known in the art. Prior to recombinant polypeptide isolation, the chloroplast may also act to protect the recombinant polypeptide from cytoplasmic degradation processes, increasing accumulation and production of the polypeptide.

Alternatively, the recombinant protein enriched chloroplast may be orally ingested. Such encapsulation will protect the recombinant polypeptide from, for example, digestive processes that may degrade the polypeptide, preventing it from performing its biological function. The foregoing feature of the methodologies of the present disclosure allows for the economic production of recombinant polypeptides in algal cells. Furthermore, the methodologies may be used for the production of recombinant polypeptides at laboratory scale and may readily be scaled up to produce the polypeptides at commercial scale bioreactors to meet the production demand for a given recombinant polypeptide.

A worker skilled in the art would readily appreciate that the methods of the invention can be used with any or all algal cells or algae including, without limitation any algae classified as cyanobacteria (Cyanophyceae), green algae (Chlorophyceae), diatoms (Bacillariophyceae), yellow-green algae (Xanthophyceae), golden algae (Chrysophyceae), red algae (Rhodophyceae), brown algae (Phaeophyceae), dinoflagellates (Dinophyceae) or pico-plankton (Prasinophyceae and Eustigmatophyceae). Examples of algal cells further include any algal species belonging to the genus, Clamydomonas, for example Chlamydomonas reinhardtii, and any algal species belonging to the genus Chlorella.

In one embodiment, the algal cell is a cyanobacteria (Cyanophyceae).

In one embodiment, the algal cell is a green algae (Chlorophyceae).

In one embodiment, the algal cell is a diatoms (Bacillariophyceae).

In one embodiment, the algal cell is a yellow-green algae (Xanthophyceae).

In one embodiment, the algal cell is a golden algae (Chrysophyceae).

In one embodiment, the algal cell is a red algae (Rhodophyceae).

In one embodiment, the algal cell is a brown algae (Phaeophyceae).

In one embodiment, the algal cell is a dinoflagellates (Dinophyceae).

In one embodiment, the algal cell is a pico-plankton (Prasinophyceae and Eustigmatophyceae).

In one embodiment, the algal cell is an algal species belonging to the genus Clamydomonas, including but not limited to Chlamydomonas reinhardtii,

In one embodiment, the algal cell is an algal species belonging to the genus Chlorella.

In some embodiments, mixtures of algal species can be used, including but not limited to species belonging to any of the aforementioned.

In some embodiments, the algal cells are transgenic algae cells that are further modified. In some embodiments, the transgenic algae cells include a transgene, vector or like that is controlled by the recombinant protein of the invention.

Recombinant Polypeptides and Polynucleotides

The present invention provides for recombinant polypeptides that can be targeted to chloroplasts in response to stress or non-homeostatic conditions.

Targeting to chloroplasts in response to stress or non-homeostatic conditions is a result of fusion of a polypeptide of interest to an oil body protein. “Oil body protein” as used herein includes all or any proteins that are naturally associated with plant oil bodies and are naturally present on the phospholipid monolayer of plant oil bodies and includes any caleosin.

In certain embodiments the targeting polypeptide is caleosin, a derivative or fragment thereof.

In some embodiments, the targeting polypeptide includes substantially the full length caleosin. In other embodiments, the targeting polypeptide includes one or more of the central domain and proline knot motif so long as the targeting domain is sufficient to target the polypeptide to the chloroplast.

The present invention provides nucleic acid sequence encoding a fusion polypeptide comprising a portion of an oil body protein to capable of targeting of the fusion polypeptide to the algal chloroplast linked to a polypeptide of interest. The nucleic acid may further include nucleic acid sequences capable of controlling expression in an algal cell.

In one embodiment, the nucleic acid encoding a sufficient portion of an oil body protein to provide targeting of the fusion polypeptide is an intact caleosin. Example nucleic acid sequences encoding caleosins that may be used include but are not limited to SEQ.ID NO: 7 to SEQ.ID NO: 12. Further oil body proteins that may be used in accordance herewith are any caleosin obtainable or obtained from an oil seed plant including, without limitation, thale cress (Arabidopsis thalania), soybean (Glycine max), rapeseed (Brassica spp.), sunflower (Heliantus annuus), safflower (Carthamus tinctorius), mustard (Brassica spp. and Sinapis alba) and maize (Zea mays). In some embodiments, the nucleic acid sequences have been codon optimized for the specific algae.

In other embodiments, the nucleic acid encoding a sufficient portion of an oil body protein to provide targeting of the fusion polypeptide is a portion of a caleosin. In some embodiments, the portion of caleosin providing targeting comprises at least central domain of a caleosin polypeptide. Example nucleic acid sequence encoding the central domain of a caleosin include but is not limited to SEQ.ID NO: 25 to SEQ.ID NO: 28 or other nucleic acid sequences encoding a central domain of a caleosin having the amino acid sequence set forth in SEQ. ID NO: 21 to SEQ.ID NO: 25.

In some embodiments, the portion of caleosin providing targeting of the fusion polypeptide comprises a caleosin proline knot motif. Examples of nucleic acid sequences encoding a proline knot motif includes the sequence set forth in SEQ.ID NO: 46 to SEQ.ID NO: 49. Examples of proline knot polypeptides are set forth in SEQ.ID NO: 42 to SEQ.ID NO: 45.

In some embodiments, the portion of the oil body protein providing targeting comprises the N-terminal domain of a caleosin. Example nucleic acid sequences encoding an N-terminal domain of a caleosin include SEQ.ID NO: 17 to SEQ.ID NO: or other nucleic acid sequences encoding a N-terminal domain of a caleosin having the amino acid sequence set forth in SEQ. ID NO: 13 to SEQ.ID NO: 16.

In some embodiments, the portion of the oil body protein providing targeting comprises the calcium binding motif within the N-terminal domain of a caleosin. Example nucleic acid sequences encoding a calcium binding motif of a caleosin N-terminal domain include SEQ. ID NO: 54 to SEQ. ID NO: 57 or other nucleic acid sequences encoding a calcium binding motif of a caleosin having the amino acid sequence set forth in SEQ. ID NO: 50 to SEQ.ID NO: 53.

In some embodiments, the portion of the oil body protein providing targeting comprises the C-terminal domain of a caleosin. Example nucleic acid sequences encoding a C-terminal domain of a caleosin include SEQ.ID NO: 33 to SEQ.ID NO: 36 or other nucleic acid sequences encoding a C-terminal domain of a caleosin having the amino acid sequence set forth in SEQ. ID NO: 30 to SEQ.ID NO: 32.

The nucleic acid encoding a recombinant polypeptide may be any nucleic acid encoding a recombinant polypeptide, including any intact polypeptide of any length, varying from several amino acids in length to hundreds amino acids in length, or any fragment or variant form of an intact recombinant polypeptide. In addition, in some embodiments, the nucleic acid encoding the polypeptide of interest may encode multiple polypeptides of interest, for example, a first and a second recombinant polypeptide, which may be linked to one another.

The recombinant polypeptide of interest may be any recombinant polypeptide including, without limitation insulin, hirudin, an interferon, a cytokine, a growth factor, an immunoglobulin or fragment thereof, an antigenic polypeptide, a hemiostatic factor, such as Willebrand Factor, a peptide hormone, such as angiotensin, β-glucuronidase (GUS), factor H binding protein, gam56, VP2, cellulase, xylanase, a protease, chymosin, chitinase, lactase or other commercially relevant enzymes.

In some embodiments, the recombinant polypeptide of interest is an enzyme that can modify the constituents of the chloroplast, for example the enzyme may modify lipid metabolism within the chloroplast.

In some embodiments, the enzyme may increase the overall amount of oil produced in the chloroplasts.

Optionally, the lipid metabolism within the chloroplast is adjusted to increase the amount of omega-3 fatty acid with the chloroplast.

In some embodiments, the protein of interest is a protein that modifies the activity of another protein or impacts gene expression.

As will readily be appreciated by those of skill in the art, depending on the nucleic acid sequence encoding the recombinant polypeptide, a wide variety of polypeptides may be selected and obtained, and the utility of the selected recombinant polypeptide may vary widely. Nucleic acid sequences encoding recombinant polypeptides may be identified and retrieved from databases such as GenBank (http://www.ncbi.nlm.nih.gov/genbank/) or nucleic acid sequences may be determined by methods such as gene cloning, probing and DNA sequencing. In accordance herewith, the nucleic acid sequence encoding the recombinant polypeptide may be selected in accordance with any and all applications for which the selected polypeptide is deemed useful. The actual nucleic acid sequence of the polypeptide of interest in accordance with the present disclosure is not limited, and may be selected as desired. In accordance herewith such recombinant polypeptides may be any polypeptides for use in pharmaceutical and biopharmaceutical or veterinary applications, any polypeptides for use in food, feed, nutritional and nutraceutical applications, any polypeptides for use in cosmetic and personal care applications, any polypeptides for use in agricultural applications, any polypeptides for use in industrial or domestic applications, any polypeptides that may be beneficial for algal growth, for example enzymes providing herbicidal or antibiotic resistance, and recombinant polypeptides for any other uses one desires to produce in accordance in accordance with the present disclosure.

In some embodiments, the 3′ end of the nucleic acid sequence encoding the sufficient portion of a polypeptide to provide targeting to a chloroplast is linked to the 5′ end of the nucleic acid sequence encoding the polypeptide of interest.

In some embodiments, the 5′ end nucleic acid sequence encoding the sufficient portion of a polypeptide to provide targeting to a chloroplast is linked to the 3′ end of the nucleic acid sequence encoding the polypeptide of interest.

In some embodiments, both the 5′ end and the 3′ end of the nucleic acid sequence encoding a sufficient portion of a polypeptide to provide targeting to a chloroplast are linked to the 3′ end a nucleic acid sequence encoding the polypeptide of interest and to the 5′ end of a nucleic acid sequence encoding a polypeptide of interest, respectively. In this embodiment, the two recombinant polypeptides of interest may be identical or different.

In some embodiments, the 3′ end of a first nucleic acid sequence encoding a sufficient portion of a polypeptide to provide targeting of a fusion polypeptide is linked to the 5′ end of a nucleic acid sequence encoding a polypeptide of interest and the 3′ end of the same nucleic acid sequence encoding a polypeptide of interest is linked to the 5′ end of a second nucleic acid sequence encoding a sufficient portion of a polypeptide to provide targeting of to a fusion polypeptide.

In some embodiments, the nucleic acid sequence encoding a sufficient portion of an oil body protein to provide targeting to a chloroplast is separated from the nucleic acid sequence encoding by a cleavable peptide linker sequence. In some embodiments, the cleavable peptide linker sequence is enzymatically cleavable, for example a linker sequence cleavable by enzymes such as thrombin, Factor Xa collagenase, or chymosin. An example of a linker sequence that may be used includes: SEQ.ID NO: 37 (encoded by SEQ.ID NO: 38). In other embodiments the cleavable peptide linker sequence is chemically cleavable, for example cyanogen bromide. In further embodiments the chimeric nucleic acid sequence further comprises a nucleic acid sequence that permits autocatalytic cleavage, for example, a nucleic acid sequence encoding chymosin or an intein (SEQ.ID NO: 39 (encoded by SEQ.ID NO: 40).

Nucleic acid sequences encoding fusion polypeptides can be prepared using any technique useful for the preparations of such nucleic acid sequences and generally involves obtaining a nucleic acid sequence encoding a sufficient portion of an oil body protein to target the fusion polypeptide, and a nucleic acid sequence encoding recombinant polypeptide of interest, for example by synthesizing these nucleic acid sequences, or isolating them from a natural source, and then linking the two nucleic acid sequences, using for example nucleic acid cloning vectors, such as the pUC an pET series of cloning vectors, microbial cloning host cells, such as Escherichia coli, and techniques such as restriction enzyme digestion, ligation, gel-electrophoresis, polymerase chain reactions (PCR), nucleic acid sequencing, and the like, which are generally known to those of skill in the art. Additional guidance regarding the preparation of nucleic acid sequences encoding fusion polypeptides including the use and cultivation of E. coli as a microbial cloning host may be found in: Green and Sambrook, Molecular Cloning, a Laboratory Manual, Cold Spring Harbor Laboratory Press, 2012 and Esposito et al., 2009 Methods Mol. Biol. 498: 31-54.

In accordance with one aspect hereof, the nucleic acid sequence encoding a fusion polypeptide is linked to a nucleic acid sequence capable of controlling expression in an algal cell. Accordingly, the present disclosure also provides, in one embodiment, a nucleic acid sequence encoding a fusion polypeptide comprising a sufficient portion of an oil body protein to provide targeting of the fusion polypeptide to a chloroplast linked to a recombinant polypeptide; and a nucleic acid sequence capable of controlling expression in an algal cell.

Nucleic acid sequences capable of controlling expression in algal cells that may be used herein include any transcriptional promoter capable of controlling expression of polypeptides in algal cells. Generally, promoters obtained from algal cells are used, including promoters associated with lipid production in algal cells. Promoters may be constitutive or inducible promoters, for example an oxygen inducible promoter. Examples of transcriptional promoters that may be used in accordance herewith include SEQ.ID NO: 41. Further nucleic acid sequence elements capable of controlling expression in an algal cell include transcriptional terminators, enhancers and the like, all of which may be included in the chimeric nucleic acid sequences of the present disclosure.

In accordance with one aspect of the present disclosure, the nucleic acids comprising a nucleic acid sequence capable of controlling expression in algal cell linked to a nucleic acid sequence encoding a fusion polypeptide comprising a sufficient portion of a caleosin to provide targeting of the fusion polypeptide to a chloroplast linked to a recombinant polypeptide, can be integrated into a recombinant expression vector which ensures good expression in the algal cell. Accordingly, the present disclosure, in a further aspect includes a recombinant expression vector comprising nucleic acids of the invention, wherein the expression vector is suitable for expression in an algal cell.

The term “suitable for expression in an algal cell”, as used herein, means that the recombinant expression vector comprises the chimeric nucleic acid sequence of the present disclosure linked to genetic elements required to achieve expression in an algal cell. Genetic elements that may be included in the expression vector in this regard include a transcriptional termination region, one or more nucleic acid sequences encoding marker genes, one or more origins of replication and the like. The genetic elements are operably linked, typically as well be known to those of skill in the art, by linking e.g. a promoter in the 5′ to 3′ direction of transcription to a coding sequence. In certain embodiments, the expression vector may further comprise genetic elements required for the integration of the vector or a portion thereof in the algal cell's genome.

Pursuant to the present disclosure, the expression vector can further contain a marker gene. Marker genes that may be used in accordance with the present disclosure include all genes that allow the distinction of transformed algal cells from non-transformed cells, including all selectable and screenable marker genes. A marker gene may be a resistance marker such as an antibiotic resistance marker against, for example, kanamycin, ampicillin, hygromycin and zeomycin. Further markers include herbicide resistance markers such as norflurazon. Screenable markers that may be employed to identify transformants through visual inspection include, β-galactosidase, β-glucuronidase (GUS) (U.S. Pat. Nos. 5,268,463 and 5,599,670) and green fluorescent protein (GFP) (Niedz et al., 1995,. Plant Cell Rep 14:403-406) and other fluorescent proteins.

To assemble the expression vector an intermediary cloning host can be used. One intermediary cloning host cell that may be used is E. coli using various techniques that are generally known to those of skill in the art including hereinbefore mentioned techniques for cloning and cultivation and general guidance that can for example be found in Sambrook et al., Molecular Cloning, a Laboratory Manual, Cold Spring Harbor Laboratory Press, 2001, Third Ed.

To introduce the chimeric nucleic acid sequence in algal cells, algal cells can be transformed using any technique known to the art, including, but not limited to, biolistic bombardment, glass beads, autolysin assisted transformation, electroporation, silicon carbide whiskers (Dunahay, T. G. (1993). BioTechniques 15, 452-460. Dunahay, T. G., Adler, S. A., and Jarvik, J. W. (1997). Methods Mol. Biol. 62, 503-509), Agrobacterium-mediated gene transfer, and sonication/ultrasonication. The selected transformation technique can be varied depending on the algal species selected. In embodiments hereof, in which the selected algal cells lack a cell wall, glass bead transformation method is preferred. In the performance of this method, in general, glass beads containing the chimeric nucleic acid sequence, for example a linearized chimeric nucleic acid sequence, are placed in a reaction tube with an algae cell suspension and the mixture is vigorously vortexed for a period of time in order to effect uptake of the chimeric nucleic acid sequence by the algal cells (Kindle, K. L., (1990). Proc. Natl. Acad. Sci (USA) 87, 1228-1232). In certain embodiments hereof in which the algal cells have cell walls, autolysin assisted transformation may be used. In general, autolysin assisted transformation methodology, involves the incubation of algal cells with autolysin, an enzyme which naturally digests the cell wall during cellular mating and renders the algal cells susceptible to the receipt of nucleic acid material (Nelson et al., Mol. Cell Biol. 14: 4011-4019). In the performance of electroporation-based techniques, an electric field is applied to the algal host cells to induce membrane permeability, in order to effect uptake by the algal cells of the chimeric nucleic acid sequence. Electroporation is a particularly preferred methodology since many algal species are readily susceptible to uptake of nucleic acid material upon electroporation (Brown et al., Mol. Cell Biol. (1991) 11 (4) 2382-2332 (PMC359944). A further methodology which in certain embodiments hereof can be used is biolistic bombardment. In the performance of biolistic bombardment-based techniques, in general, a particle delivery system is used to introduce the chimeric nucleic acid sequence into algae cells (Randolph-Anderson et al., BioRad Technical Bulletin no 2015 [http://www.bio-medicine.org/biology-technology/Sub-Micron-Gold-Particles-Are-Superior-to-Larger-Particles-for-Efficient-Biolistic-Transformation-of- Organelles-and-Some-Cells-1201-1/]. A further methodology that can be used to obtain transformed algal cells is Agrobacterium tumefaciens mediated transformation, which in general involves the infection of algal cells with Agrobacterium cells transformed to contain the chimeric nucleic acid sequence and upon infection transfer of the chimeric nucleic acid sequence to algal cells (Kumar, S. V. et al. (2004). Genetic transformation of the green alga Chlamydomonas reinhardtii by Agrobacterium tumefaciens. Plant Sci. 166, 731-738). Yet one further methodology that in certain embodiments can be used is the use of ultrasound mediated delivery of the chimeric nucleic acid sequence into algae as is for example described in Unites States Patent Application no. US2015/0125960.

In some embodiments, upon introduction of the nucleic acid, the nucleic acid sequence may be incorporated in the genome of the algal cell, generally resulting in inheritable expression. In order to facilitate integration in the genome of the algal cell, the nucleic acid sequence may comprise one or more nucleic acid sequences that facilitate integration of the chimeric nucleic acid sequence in the algal genome.

In some embodiments, upon introduction of the nucleic acid sequence, the chimeric nucleic acid sequence may be maintained as a nucleic acid sequence outside of the genome of the algal cell, generally resulting in transient expression.

Growth Conditions

In order to target the fusion protein to the chloroplasts, the algal cells comprising the fusion proteins are subjected to stress or non-homeostatic growth condition.

In accordance with certain embodiments hereof, the algal cell is grown in a growth medium under non-homeostatic growth conditions to target the recombinant polypeptide to the chloroplast within the algal cell.

In some embodiments, the algal cell is during a first time period grown under homeostatic growth conditions wherein during such first time period substantially no chloroplastic-targeting occurs, and during a second time period grown under conditions under non-homeostatic growth conditions.

Growth of algal cells under homeostatic conditions can be performed using any growth media suitable for the growth of algal cells, comprising non-limiting amounts of nutrients, including nutrients providing a carbon source, a nitrogen source, and a phosphorus source, as well as trace elements such as aluminum, cobalt, iron, magnesium, manganese, nickel, selenium zinc, and the like, and growing algal cells under optimal growth conditions. Conditions to achieve homeostatic growth for algal cells vary depending on the selected algal species, however such conditions typically include temperatures ranging, from 20° C. to 30° C., light intensities varying from 25-150 μE m⁻² s⁻¹ and a pH that is maintained in a range from 6.8 to 7.8.

Homeostatic growth conditions include conditions appropriate for batch cultivation of algal cells, as well as conditions for continuous algal cell cultivation. In some embodiments liquid culture media are used to grow the algal cells. In alternate embodiments, solid media for algal growth may also be used as a substrate for algal growth (The Chlamydomonas Sourcebook (Second Edition) Edited by:Elizabeth H. Harris, Ph.D., David B. Stern, Ph.D., and George B. Witman, Ph.D. ISBN: 978-0-12-370873-1) Further guidance to prepare suitable media for the homeostatic growth of algae, as well as guidance to suitable culturing conditions for algae are further described in Appl Microbiol Biotechnol. 2014 June; 98 (11):5069-79. doi: 10.1007/s00253-014-5593-y. Epub 2014 Mar. 4; Handbook of Microalgal Culture: Applied Phycology and Biotechnology By Amos Richmond, Qiang Hu ISBN 140517249; and in Algal Culturing Techniques Robert Arthur Anderson 2005 ISBN 0120884267. The concentration of a nutrient and/or a growth condition may be optimized or adjusted, for example by preparing a plurality of growth media, each including a different concentration of a nutrient, growing algal cells in each of the growth media, and evaluating algal growth, example, by evaluating cell density as a function of time. Then, a growth medium or growth condition can be selected that provides the most desirable effect.

In accordance with one embodiment, the algal cells are subjected to non-homeostatic conditions. By “subjecting to non-homeostatic conditions”, it is meant that the conditions under which the algal cells are grown are gradually or abruptly modulated or established in such a manner that algal cell growth rates substantially deviate from growth rates under homeostatic growth conditions. Thus, for example, the algal cell growth rate during log phase growth under homeostatic growth conditions deviates substantially from the algal cell growth rate during log phase growth under non-homeostatic conditions, and the algal cell growth rate during stationary phase growth under homeostatic growth conditions deviates substantially from the algal cell growth rate under non-homeostatic conditions. Substantial deviations include deviations wherein the growth rate under a non-homeostatic condition is less than about 0.8 or 0.8, about 0.7 or 0.7, about 0.6 or 0.6, about 0.5 or 0.5, about 0.4 or 0.4, about 0.3 or 0.3, about 0.2 or 0.2, or about 0.1 or 0.1 times the growth rate under a corresponding homeostatic growth condition. The aforementioned condition change may be brought about by several different means. In one non-limiting example, one skilled in the art may replace regular growth media with another growth media intended to provide a desirable effect. Another non-limiting example is abstaining from supplementing the culture with additional nutrients, resulting in the culture's own gradual consumption of nutrients, modulating growth conditions to a non-homeostatic state.

In some embodiments, the algal cells immediately following introduction of the nucleic acid within the algal cells are grown under non-homeostatic conditions. In some embodiments, the cells are grown or maintained in lag phase and not permitted to proceed from growth to logarithmic phase.

In some embodiments, the algal cells during a first time period, for example immediately following the introduction of the chimeric nucleic acid sequence, are grown under homeostatic conditions, and are then subjected to non-homeostatic conditions to grow or maintain the algal cells during a second time period under non-homeostatic conditions. In one embodiment, the algal cells are during a first time period grown to logarithmic phase, and while in logarithmic phase the cells are subjected to non-homeostatic growth conditions to grow or maintain the algal cells under non-homeostatic conditions during a second time period. Thus in this embodiment, the doubling rate decreases from a logarithmic doubling rate to a doubling rate that is substantially lower than the doubling rate under logarithmic homeostatic conditions, for example, the doubling rate under non-homeostatic conditions is less than about 0.8 or 0.8, about 0.7 or 0.7, about 0.6 or 0.6, about 0.5 or 0.5, about 0.4 or 0.4, about 0.3 or 0.3, about 0.2 or 0.2, or about 0.1 or 0.1 times the doubling rate under homeostatic growth conditions during logarithmic phase. In some embodiments the doubling rate, upon subjecting the cells to non-homeostatic conditions may alter from a constant doubling rate to a declining doubling rate. In some embodiments, upon subjecting the cells to non-homeostatic conditions, the cells may enter a different growth phase, for example the cells may upon being subjected to non-homeostatic growth conditions enter the stationary growth phase from logarithmic phase.

In one embodiment, non-homeostatic growth conditions are conditions in which one or more nutrients are present in the algal cell growth medium in quantities that are insufficient for homeostatic algal cell growth.

In one embodiment, non-homeostatic growth conditions are conditions in which nitrogen is present in the algal cell growth medium in quantities that are insufficient for homeostatic algal cell growth. In some embodiments, the quantities of nitrogen present in the medium to for non-homeostatic growth ranges from about 0 mole/liter to about 0.02 mole/liter

In one embodiment, non-homeostatic growth conditions are conditions in which phosphorus is present in the algal cell growth medium in quantities that are insufficient for homeostatic algal cell growth. In some embodiments, the quantities of phosphorus present in the medium for non-homeostatic growth ranges from about 0 to 0.8 mM.

In another embodiment, an exogenous stress factor, for example a physical, chemical or biological stress factor, is applied to an algal cell culture comprising a chimeric nucleic acid sequence of the present disclosure to effect non-homeostatic conditions.

In one embodiment, the exogenous stress factor applied is an adjustment of the pH of an algal cell culture to obtain a growth medium having non-homeostatic pH and growing the cells at a non-homeostatic pH. In some embodiments, the pH is adjusted in such a manner that the pH of the algal culture ranges between about pH 5.0 to 6.5.

In one embodiment, the exogenous stress factor applied is an adjustment of the salinity of an algal cell culture to obtain a growth medium having a non-homeostatic salinity and growing the cells under non-homeostatic salinity. In some embodiments, the salinity is adjusted in such a manner that the concentration of sodium and chloride ions of the algal culture ranges between about 20 to about 200 mM.

In one embodiment, the exogenous stress factor applied is an adjustment of the light intensity to which an algal cell culture is exposed to obtain a growth condition having a non-homeostatic light intensity and growing the cells under non-homeostatic light intensity. In some embodiments, the light intensity is adjusted in such a manner that the light intensity to which the algal culture is exposed ranges between about 150-1000 μE m⁻² s⁻¹.

Non-homeostatic growth conditions may be detected and measured by comparing growth of algal cells under homeostatic conditions with growth of algal cells under non-homeostatic conditions. Thus, for example, the cell density of an algal cell culture may be determined, for example, by determining the optical density, or a cell counter such as a Coulter counter or flow cytometrically, and the densities of algal cell cultures grown under homeostatic and non-homeostatic growth conditions may be compared. By measuring the cell density at different time points the growth rate and doubling rate of an algal cell culture, whether grown under homeostatic or non-homeostatic conditions, may be determined. Further guidance with respect to measuring algal cell growth may be found in The Chlamydomonas Sourcebook (Second Edition) Edited by: Elizabeth H. Harris, Ph.D., David B. Stern, Ph.D., and George B. Witman, Ph.D. ISBN: 978-0-12-370873-1)

In accordance with one aspect hereof, upon growth under non-homeostatic conditions, the fusion polypeptide comprising the recombinant polypeptide is targeted and accumulated in the algal chloroplast.

In accordance with one embodiment, the newly-synthesized polypeptide is associated with lipids throughout the algal cell, and upon growth under non-homeostatic conditions, target to the algal chloroplasts. Production of lipids, including in association with chloroplasts and recombinant polypeptides may be evaluated by staining algal cells with a lipophilic stain, such as Nile Red.

In accordance with one embodiment, hereof the fusion polypeptide is produced in association with the algal chloroplasts and the fusion polypeptide is protected from exposure to the cytoplasm, and from degradation by cytoplasmic enzymes.

In some embodiments, targeting of the fusion polypeptide may be evaluated, for example using techniques such as electron microscopy, and confocal fluorescent microscopy in conjunction with fluorescent antibodies having a specificity for the recombinant polypeptide of interest.

In some embodiments, the fusion protein includes a detectable tag, for example, a fluorescent tag.

In different embodiments, the algal cells may be subject to different non-homeostatic conditions, as herein before described, for example, in the presence of quantities of nutrients, such as nitrogen, or phosphate in quantities that are insufficient for homeostatic growth, or by subjecting the cells to an exogonous stress factor e.g. non-homeostatic pH conditions, non-homeostating light conditions or non-homeostatic salinity etc.

In some embodiments, the recombinant algal cells are grown at 22° C. and CO₂ over 0.5% to facilitate clumping.

Harvesting

In accordance with some embodiments the algal cells may be harvested, and the chloroplasts may be isolated from the algal cell, as hereinbefore described.

Algal cells may be harvested by a variety of techniques known in the art including centrifugation and filtration. Optionally, harvesting includes a flocculation step where clumping of algal cells is promoted by growth conditions and/or additives and/or other methods known in the art.

In accordance with some embodiments where the harvesting of algal cells includes a flocculation step, the algal clumps are isolated.

In some embodiments after the algal cells are harvested, chloroplasts are isolated.

In accordance with one embodiment, chloroplasts may be isolated from the algal cells. Methodologies for the isolation of chloroplasts from algal will generally be known to those of skill in the art and include but are not limited to the methodologies described in Mason, et al. (2006). Nat. Protoc. 1, 2227-2230. The chloroplasts thus isolated comprise a fusion polypeptide comprising a sufficient portion of an oil body protein to provide targeting to a chloroplast and a recombinant polypeptide.

In accordance with one embodiment, the fusion polypeptide may include a cleavable linker sequence and upon isolation of the chloroplasts the recombinant polypeptide may be separated from the chloroplasts and the oil body protein, or portion thereof, as the case may be, and a substantially pure recombinant polypeptide may be obtained, using any protein purification methodology, including without limitation, those hereinbefore described.

### Example

Hereinafter are provided examples of specific implementations for performing the methods of the present disclosure, as well as implementations representing the compositions of the present disclosure. The examples are provided for illustrative purposes only, and are not intended to limit the scope of the present disclosure in any way.

Vector Construction

The pChlamy_3 plasmid was obtained from Invitrogen. All standard recombinant DNA techniques (DNA digestion by restriction endonucleases, DNA ligation, plasmid isolation, and preparation of media and buffers) were performed as previously described (Sambrook, Fritsch, & Maniatis, 1989). The restriction endonucleases BamHI, Kpnl, Xbal, Xhol, and T4 DNA ligase were from New England Biolabs.

Transformation of Algal Cells

This method was used to transform a particular strain of Chlamydomonas reinhardtii via electroporation to introduce the caleosin into the organism. The strains and culture conditions are as follows. Wild type C. reinhardtii cells of strain mt-[137c] were obtained from the Chlamydomonas Research Center (St. Paul, Minn.). Cells were grown at room temperature (RT) (22° C.) on a gyratory shaker at 120 rpm at a light intensity of 50 uE m-2 s-1 and a starting cell density of approximately 1.0×10⁵ cells/mL. Cells were grown in Tris-Acetate-Phosphate (TAP) culture medium (Harris, 1989).

Electroporation of Chlamydomonas cells with plasmid DNA was performed as previously described (Invitrogen, 2013). Briefly, 2 μg plasmid DNA was mixed in a 4 mm electroporation cuvette with 5.4×10⁴ wild type C. reinhardtii cells in exponential growth and incubated at room temperature for 5 minutes. After incubation, plasmid DNA was electroporated into Chlamydomonas cells with settings 50 uF, 1.5 kV cm-1, and infinite resistance. After electroporation, cells were resuspended in 12 mL of TAP+40 mM sucrose and incubated for 24 h at RT under white LED panels of intensity 50 uE m-2 s-1 with agitation of 100 rpm. After recovery, cells were centrifuged for 7 min at 1200 g, and resuspended in 750 μL TAP+40 mM sucrose. 250 ul of the cells were plated on each of three TAP+selection (10 μg/L hygromycin)+1.5% agar plates and incubated right side up at RT under white LED lights of 50 uE m-2 s-1 for 5 d or until colonies are clearly visible. Single colonies of at least 2 mm in diameter were used to inoculate TAP+selection (2 μM norflurazon or 10 μg/L hygromycin as appropriate) liquid media. Liquid cultures were incubated under standard growth conditions (50 uE m-2 s-1 from white LED panels, agitated on a gyratory shaker at 120 rpm, room temperature (RT)) until desired cell density was achieved, at which point cells could be subcultured.

Chloroplast Targeting

Algal cells were inoculated in 50 mL of TAP media at a density of 1×10⁵ cells/mL and grown to late log phase. Cultures to be nitrogen stressed (grown in TAP-N) were pelleted (1200 g, 7 min) and resuspended in an equal volume TAP-N medium (Siaut 2011, BMC Biotechnology 2011 Jan. 21; 11:7 doi 10.1186/1472-6750-11-7). Control cultures (grown in TAP+N) were pelleted (1200 g, 7 min) and resuspended in an equal volume fresh TAP medium. All cultures were incubated 5 days after resuspension under standard growth conditions (50 uE m-2 s-1 from white LED panels, agitated on a gyratory shaker at 120 rpm, RT) before imaging.

To image the algal cells for targeting, 10 μl of the cultured cells of interest were transferred to a coated slide. The slide coat consisted of 2% agarose (Thermo Fisher Scientific) in TAP or TAP-N medium as appropriate, with 0.0001% w/v Nile Red (Sigma-Aldrich) added when detection of triacylglycerides was desired. A 24×40 mm (No. 1 1/2) cover glass (Corning) was placed on top of the agarose gel and sample. The edges of the slide were sealed with clear nail polish (N.Y.C.) and allowed to dry before the slide was subjected to analysis. An Olympus Fluoview FV10i (Olympus Canada Inc., Richmond Hill, Ontario) laser scanning confocal microscope was used to observe and capture images of the cells. All images were captured using an Olympus UPlanSApo 60× oil immersion objective (Olympus Canada Inc., Richmond Hill, Ontario). Additional digital magnification of 8× (total magnification of 480×) was applied using the Fluoview FV10i 1.2a software. Laser excitation and emission wavelengths for yellow fluorescent protein (YFP) were set to 480 nm and 527 nm respectively. Laser excitation and emission wavelengths for detection of triacylglycerides (TAGs) stained with Nile Red (NR) were set to 533 nm and 574 nm respectively. Where applicable, chloroplast autofluorescence (CHL) was imaged using an excitation wavelength of 473 nm and emission wavelength of 670 nm.

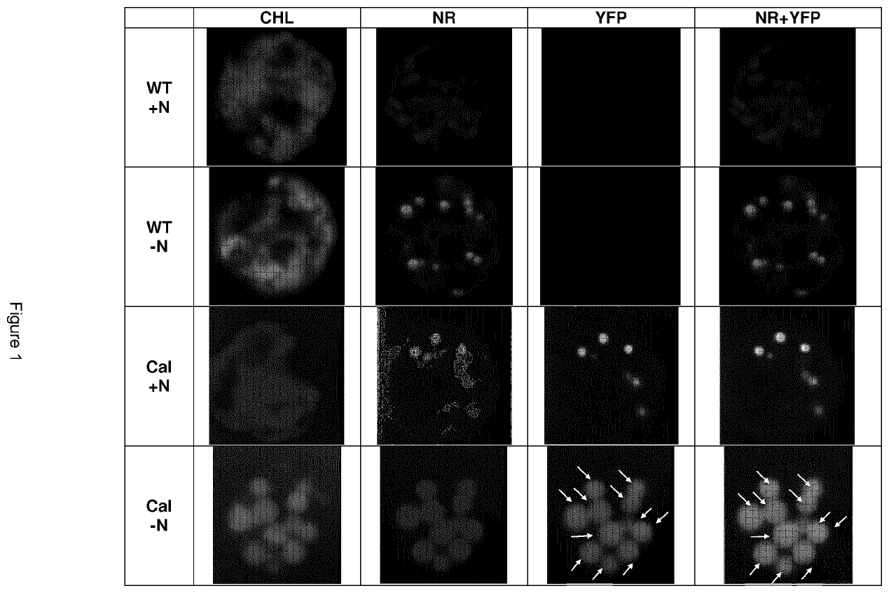

Referring to FIG. 1, Caleosin-YFP is targeted to the cytoplasm under homeostatic conditions (normal nitrogen levels, see row “Cal+N”, all panels) while under stress (nitrogen depletion, see row “Cal−N”, all panels) Caleosin-YFP is targeted to the chloroplast. Areas of Caleosin-YFP accumulation are indicated by white arrows in panels YFP and YFP+NR of row “Cal−N”. Caleosin-YFP is located in the same areas as Nile Red under normal and depleted nitrogen levels (Rows Cal+N and Cal−N, column NR+YFP). Caleosin-YFP is located in the same areas as the chloroplast only under non-homeostatic conditions (Row Cal−N, comparing column CHL and YFP). Comparative panels of wild type algae (WT+N, WT-N) are provided for comparison.

SEQUENCE TABLE

| SEQUENCE | | |
|---|---|---|
| IDENTIFIER | SEQUENCE | NOTES |
| SEQ. ID NO: 1 | MGSKTEMMERDAMATVAPYAPVTYHRRARVDLDDR | Arabidopsis thaliana |
| | LKPYMPRALQAPDREHPYGTPGHKNYGLSVLQQH | Caleosin |
| | VSFFDIDDNGIIYPWETYSGLRMLGFNIIGSLIIAAVINL | |
| | TLSYATLPGWLPSPFFPIYIHNIHKSKHGSDSKTYDNE | |
| | GRFMPVNLELIFSKYAKTLPDKLSLGELWEMTEGNR | |
| | DAWDIFGWIAGKIEWGLLYLLARDEEGFLSKEAIRRC | |
| | FDGSLFEYCAKIYAGISEDKTAYY | |
| SEQ. ID NO: 2 | MAEEAASKAAPTDALSSVAAEAPVTRERPVRADLEV | Arachis hypogaea |
| | QIPKPYLARALVAPDVYHPEGTEGRDHRQMSVLQQH | Caleosin |
| | VAFFDLDGDGIVYPWETYGGLRELGFNVIVSFFLAIAI | |
| | NVGLSYPTLPSWIPSLLFPIHIKNIHRAKHGSDSSTYD | |
| | NEGRFMPVNFESIFSKNARTAPDKLTFGDIWRMTEG | |
| | QRVALDLLGRIASKGEWILLYVLAKDEEGFLRKEAVR | |
| | RCFDGSLFESIAQQRREAHEKQK | |
| SEQ. ID NO: 3 | MATHVLAAAAERNAALAPDAPLAPVTMERPVRTDLE | Sesamum indicum |
| | TSIPKPYMARGLVAPDMDHPNGTPGHVHDNLSVLQQ | Caleosin |
| | HCAFFDQDDNGIIYPWETYSGLRQIGFNVIASLIMAIVI | |
| | NVALSYPTLPGWIPSPFFPIYLYNIHKAKHGSDSGTYD | |
| | TEGRYLPMNFENLFSKHARTMPDRLTLGELWSMTEA | |
| | NREAFDIFGWIASKMEWTLLYILARDQDGFLSKEAIR | |
| | RCYDGSLFEYCAKMQRGAEDKMK | |
| SEQ. ID NO: 4 | MASNESLQTTAAMAPVTIERRVNPNLDDELPKPFLPR | Pinus massoniana |
| | ALVAVDTEHPSGTPGHQHGDMSVLQQHVAFSNRNN | Caleosin |
| | DGIVYPWETFLGFRAVGFNIIISFFGCLIINIFLSYPTLP | |
| | GWIPSPFFPIYIDRIHRAKHGSDSEVYDTEGRFVPAKF | |
| | EEIFTKNAKTHPDKLSFSELWNLTEHNRNALDPLGWI | |
| | AAKLEWFLLYSLAKDPHGFVPKEAARGVFDGSLFEF | |
| | CEKSRKVKQATVKSLTFKI | |
| SEQ. ID NO: 5 | MSTATEIMERDAMATVAPYAPVTFHRRARVDMDDRL | Brassica napus |
| | PKPYMPRALQAPDREHPYGTPGHKNYGLSVLQQHV | Caleosin |
| | AFFDLDDNGIIYPWETYSGLRMLGFNIIVSLIAAAVINL | |
| | ALSYATLTGWFPSPFFPIYIHNIHKSKHGSDSRTYDNE | |
| | GRFMPVNLELIFSKYAKTLPDKLSLGELWEMTQGQR | |
| | DAWDIFGWFASKIEWGLLYLLARDEEGFLSKEAIRRC | |
| | FDGSLFEYCAKIYAGINEDKTAYY | |
| SEQ. ID NO: 6 | MEVGRTPRRRASPAAAAAAAAAAVPSLLLFAVLFVG | Zea mays |
| | RAAAALGGPGPALYKHASFFDRDGDGVVSFAETYGA | Caleosin |
| | FRALGFGLGLSSASAAFINGALGSKCRPQNATSSKLD | |
| | IYIEDIRRGKHGSDSGSYDAQGRFVPEKFEEIFARHA | |
| | RTVPDALTSDEIDQLLQANREPGDYSGWAGAEAEW | |
| | KILYSLGKDGDGLLRKDVARSVYDGTLFHRLAPRWK | |
| | SPDSDMERS | |
| SEQ. ID NO: 7 | AAAGTGAGAGAGAGATGGGGTCAAAGACGGAGAT | Arabidopsis thaliana |
| | GATGGAGAGAGACGCAATGGCTACGGTGGCTCCC | Caleosin |
| | TATGCGCCGGTCACTTACCATCGCCGTGCTCGTGT | |
| | TGACTTGGATGATAGACTTCCTAAACCTTATATGCC | |
| | AAGAGCATTGCAAGCACCAGACAGAGAACACCCG | |
| | TACGGAACTCCAGGCCATAAGAATTACGGACTTAG | |
| | TGTTCTTCAACAGCATGTCTCCTTCTTCGATATCGA | |
| | TGATAATGGCATCATTTACCCTTGGGAGACCTACT | |
| | CTGGACTGCGAATGCTTGGTTTCAATATCATTGGG | |
| | TCGCTTATAATAGCCGCTGTTATCAACCTGACCCTT | |
| | AGCTATGCCACTCTTCCGGGGTGGTTACCTTCACC | |
| | TTTCTTCCCTATATACATACACAACATACACAAGTC | |
| | AAAGCATGGAAGTGATTCAAAAACACATGACAATG | |
| | AAGGAAGGTTTATGCCGGTGAATCTTGAGTTGATA | |
| | TTTAGCAAATATGCGAAAACCTTGCCAGACAAGTT | |
| | GAGTCTTGGAGAACTATGGGAGATGACAGAAGGA | |
| | AACCGTGACGCTTGGGACATTTTTGGATGGATCGC | |
| | AGGCAAAATAGAGTGGGGACTGTTGTACTTGCTAG | |
| | CAAGGGATGAAGAAGGGTTTTTGTCAAAAGAAGCT | |
| | ATTAGGCGGTGTTTCGATGGAAGCTTGTTCGAGTA | |
| | CTGTGCCAAAATCTACGCTGGTATCAGTGAAGACA | |
| | AGACAGCATACTACTAAAAGTATCCTTTATGTTAAG | |
| | TAATTGATCGAGCCATTTTAAGCTAATAATCGATCA | |
| | ATGTGAAGCTTGTGCCTATACGGTAAATGAAGGTT | |
| | CGGGTAGTAGTATGGACTTTTGGTCTAAGAGATCT | |
| | ATGTTTGTTTTTGTTTTTCCAGTTCTGTATGGTTATA | |
| | CTATAAGTTGCAGCTCTAAAGAAAAGCTTCTGTATG | |
| | TTTTGTTGCCTTGGTCTCTCTTTGTACCAACCCCTT | |
| | TTTCTGTTATTTCCAATTTTACACTGTTAGTTATTAT | |
| | TGCTGAAAAAAAAAAAA AAAA | |
| SEQ. ID NO: 8 | GCAATTTTGCAAAGCGAGAAATTCCACACAGGTTA | Arachis hypogaea |
| | CACCAGTATATACGCATCTTGCTAAACCGACTACT | Caleosin |
| | GATCGAGATCGCTATGGCGGAGGAGGCGGCTAGC | |
| | AAGGCAGCGCCGACCGATGCGCTGTCGTCCGTGG | |
| | CGGCGGAGGCGCCGGTGACGAGAGAACGGCCGG | |
| | TCCGAGCGGACTTGGAAGTGCAGATTCCGAAGCC | |
| | CTATTTGGCCCGAGCTCTGGTTGCTCCGGACGTGT | |
| | ACCATCCTGAAGGAACCGAGGGGCGTGACCACCG | |
| | GCAGATGAGTGTGCTGCAGCAGCATGTGGCTTTCT | |
| | TCGACCTGGATGGCGACGGTATCGTTTATCCATGG | |
| | GAAACTTATGGAGGACTACGGGAATTGGGCTTCAA | |
| | CGTGATTGTTTCGTTCTTTTTGGCGATAGCCATAAA | |
| | CGTTGGTCTAAGCTACCCAACTCTGCCAAGCTGGA | |
| | TACCATCTCTCCTGTTCCCTATACACATAAAAAACA | |
| | TCCACAGGGCTAAGCACGGCAGCGATAGCTCGAC | |
| | GTACGACAACGAGGGAAGGTTTATGCCGGTCAATT | |
| | TCGAGAGCATCTTCAGCAAGAACGCCCGCACGGC | |
| | GCCGGACAAGCTCACGTTCGGCGATATCTGGCGG | |
| | ATGACCGAAGGCCAAAGGGTGGCGCTCGACTTGC | |
| | TTGGGAGGATCGCGAGTAAGGGGGAGTGGATATT | |
| | GCTCTACGTGCTTGCGAAAGATGAGGAAGGATTCC | |
| | TCAGGAAGGAGGCTGTTCGCCGCTGCTTCGATGG | |
| | GAGCCTATTCGAGTCGATTGCCCAGCAGAGAAGG | |
| | GAGGCACATGAGAAGCAGAAGTAGCCTCCTAATTT | |
| | CATCGTCCCGGGACCTGGGATGTGCTTGATTGCTT | |
| | GTGTGTGTTGTTGTGTGGACTATAGCTATAGCCAC | |
| | ATCATGTTTGTCCATCTGAAAAAACAATGGAAATAA | |
| | GGTTTACCGGTTGGAACATACATTATGTACTATCCA | |
| | TGTGATTATTGAAATGTGTCTGTAACCTGAAAGTGT | |
| | GATTGACATATAAAATTCTGTGATTGAAGTAAAGGT | |
| | AAGCATTAAAAAAAAAA AAAAAAA | |
| SEQ. ID NO: 9 | GGCACGAGAGAGAAAAAAGGTGATTTTGTCAAGG | Sesamum indicum |
| | GAAATATGGCAACTCATGTTTTGGCTGCTGCGGCG | Caleosin |
| | GAGAGAAATGCTGCGTTGGCGCCGGACGCCCCGC | |
| | TTGCTCCGGTGACTATGGAGCGCCCAGTGCGCAC | |
| | TGACTTGGAGACTTCGATCCCGAAGCCCTATATGG | |
| | CAAGAGGATTGGTTGCACCTGATATGGATCACCCC | |
| | AACGGAACACCAGGCCATGTGCATGATAATTTGAG | |
| | TGTGCTGCAACAGCATTGTGCTTTCTTTGATCAGG | |
| | ATGATAACGGAATCATCTATCCATGGGAGACTTAC | |
| | TCTGGACTTCGCCAAATTGGTTTCAATGTGATAGCT | |
| | TCCCTTATAATGGCTATCGTCATTAATGTGGCGCT | |
| | GAGTTATCCTACTCTCCCGGGTTGGATTCCTTCTC | |
| | CTTTTTTCCCCATATATTTGTACAACATACACAAGG | |
| | CCAAACATGGAAGCGACTCCGGAACCTATGATACT | |
| | GAAGGAAGGTACCTACCTATGAATTTTGAGAACCT | |
| | GTTCAGCAAGCATGCCCGGACAATGCCCGATAGG | |
| | CTCACTCTAGGGGAGCTATGGAGCATGACTGAAG | |
| | CTAACAGAGAAGCATTTGACATTTTCGGCTGGATC | |
| | GCAAGCAAAATGGAGTGGACTCTCCTCTACATTCT | |
| | TGCAAGAGACCAGGACGGTTTCCTGTCGAAAGAA | |
| | GCCATCAGGCGGTGTTACGATGGCAGTTTGTTCGA | |
| | GTACTGTGCAAAGATGCAAAGGGGAGCCGAGGAC | |
| | AAGATGAAATGAAGGAAATCGGCTATCGCGGTAGG | |
| | TGTAAGTTATGATGTGGTGTGTATGATGGATTGAAA | |
| | GTGCCAGTGCTTAAGTTGTGTGGCAGAGTCTTGTG | |
| | TAATAACCTTTGTGTACAGATTTAAGGTCTCGGAAT | |
| | TGGTGTAACTGTGGAGAAGATGTTGACTCCTGTTT | |
| | TTGTTCAATAAGTCCAACTCTTGACATTTGGTTGGT | |
| | TTGCAGGGAAAGATGGGGAATTTTGTTTTCCGAAA | |
| | AAAAAAAAAAAAAAAAA | |
| SEQ. ID NO: 10 | ATGGGGGTGCTGCAAAAAAAATTGAACTTCATCAA | Pinus massoniana |
| | ATCTAGTTCCAGGAATTGTAGGTCGCGAGGTCGGA | Caleosin |
| | TCTGTGGGACTGAGCAAATTATTATCACTGTGATC | |
| | GAGAAAGCATTTAAGTACCAGCTATAATGGCTTCC | |
| | AATGAATCTTTACAGACAACAGCTGCTATGGCACC | |
| | AGTAACAATCGAGCGCAGGGTTAACCCCAATCTCG | |
| | ATGACGAACTCCCAAAACCTTTTCTCCCAAGGGCG | |
| | CTCGTAGCAGTTGACACAGAACATCCGAGTGGAAC | |
| | CCCTGGACACCAACACGGCGACATGAGCGTTCTT | |
| | CAACAGCACGTCGCATTTTCCAATCGCAACAACGA | |
| | CGGGATTGTGTACCCTTGGGAGACTTTCTTAGGTT | |
| | TTCGTGCCGTGGGTTTTAATATAATAATCTCGTTCT | |
| | TTGGTTGCCTTATTATCAACATTTTCTTGAGCTATC | |
| | CTACGTTGCCTGGATGGATTCCCTCGCCATTTTTT | |
| | CCAATCTATATTGATAGGATTCATCGAGCGAAGCA | |
| | TGGAAGCGATTCCGAAGTTTATGACACAGAAGGAA | |
| | GGTTTGTCCCCGCTAAATTCGAAGAAATTTTTACAA | |
| | AAAATGCCAAAACCCATCCAGATAAACTGTCATTCT | |
| | CTGAGCTGTGGAATTTGACGGAACACAATAGAAAT | |
| | GCGCTTGATCCTTTAGGATGGATTGCGGCGAAGTT | |
| | AGAATGGTTCTTGTTATACTCTCTGGCTAAAGACCC | |
| | CCATGGTTTTGTGCCCAAGGAAGCTGCGAGAGGT | |
| | GTATTTGATGGTAGCTTGTTCGAGTTCTGCGAGAA | |
| | GTCTCGAAAGGTCAAACAAGCAACAGTGAAATCCC | |
| | TGACCTTTAAGATTTGAAGCTCTAAAAACTCTTGCG | |
| | GTCATTGTCATAAATTGGTGCTCTCTTTATGTCTAT | |
| | AAGGTGGACTACTCTACAAGATGGGCTGCCATGTA | |
| | TATATAGGAAGATATGCATTGAAGTAGGAATCAACT | |
| | GGTTGAGCCTCTTCTAGATGGAAGATTGTAGAGTC | |
| | ATGAAACCTCCCTCCCATATAAGTAAGACAATATTA | |
| | GTCAGAAGAGAGAAAAATCTCTGCGTGATACCACT | |
| | GCTGCCTAAAGAAGTCGATTAGAATCACTAGTGAT | |
| | CGCGCCGCTGCAGTCGAACATATGGGAAGCTCCC | |
| | ACCGTGAT GCAAGCTGA | |
| SEQ. ID NO: 11 | TACGGCCGGGGATTGCACTCGGTCCACAGAGCAA | Brassica napus |
| | GAAAGAGCGAGAGATGAGTACGGCGACTGAGATA | Caleosin |
| | ATGGAGAGAGACGCAATGGCTACGGTGGCTCCCT | |
| | ACGCTCCGGTCACCTTTCACCGCCGTGCTCGTGTT | |
| | GACATGGATGATAGACTTCCTAAACCTTATATGCCA | |
| | AGAGCACTGCAAGCACCCGACAGAGAGCATCCGT | |
| | ATGGAACCCCAGGCCATAAGAATTATGGACTTAGT | |
| | GTTCTTCAGCAACATGTCGCCTTCTTCGATTTAGAT | |
| | GATAATGGAATTATCTATCCTTGGGAGACCTACTCT | |
| | GGACTGCGAATGCTAGGTTTCAATATCATTGTATC | |
| | GCTTATCGCAGCCGCTGTAATCAACTTGGCCCTTA | |
| | GCTATGCTACTCTTACGGGATGGTTTCCTTCGCCG | |
| | TTCTTCCCAATATACATACACAATATACACAAGTCA | |
| | AAGCATGGGAGCGACTCAAGAACATATGACAATGA | |
| | AGGGAGGTTTATGCCTGTGAATCTTGAGTTGATAT | |
| | TTAGCAAATATGCGAAAACATTGCCAGACAAGTTG | |
| | AGTCTTGGAGAATTATGGGAGATGACACAAGGACA | |
| | ACGTGACGCATGGGACATCTTCGGATGGTTCGCAA | |
| | GCAAAATAGAGTGGGGGTTGTTGTACTTGCTAGCG | |
| | AGGGATGAAGAAGGGTTTCTGTCAAAAGAAGCGAT | |
| | TAGGAGGTGTTTTGACGGGAGCTTGTTCGAGTATT | |
| | GTGCCAAGATCTACGCAGGTATCAATGAAGACAAG | |
| | ACAGCCTACTACTAAAAGTAAATGATAGAGGAGCT | |
| | TTAGGCTGATAATCGTCCATGTGAATGTAACTTGTG | |
| | TCTAAAGCAGAGTCCATGTGTTTGTTATGTTATGTC | |
| | CAAATCTGTAAGGTAGAGTATCATCAGTTGCAGCT | |
| | GGTATAGAAAGCTTCTATGATCATAATATAGTATGT | |
| | TTGTGTGGGTTGTGTTGGGTTGATCACCCTTTTCA | |
| | GTATTCAGGTCAATGTATTTTCATGGTGTAGAGGAA | |
| | AAAAAAAAAAA | |
| SEQ. ID NO: 12 | AAGCTGCGCTGCCAGTGCCAGCGCTCACTCGAAC | Zea mays |
| | GCCGAGACCCGAGAGGAGCAAACAGCCAAAAAGA | Caleosin |
| | ACGGAAAGGGGAGAGCAAACAGCCAAAAAAGGAC | |
| | GGACTTGCGCGACAGGGTCGAAGACTCAGAAGGG | |
| | GAATCTCCGGAGGATGGAGGTGGGCAGGACTCCG | |
| | CGGCGACGGGCGTCCCCAGCGGCAGCGGCGGCG | |
| | GCGGCGGCGGCGGCTGTGCCTTCGCTGCTTCTGT | |
| | TCGCCGTGCTATTCGTGGGCCGGGCGGCGGCAG | |
| | CGTTGGGCGGCCCGGGGCCGGCGCTATACAAGC | |
| | ACGCGTCGTTCTTCGACCGCGACGGCGACGGCGT | |
| | CGTCTCCTTCGCGGAGACGTACGGCGCGTTTCGG | |
| | GCCCTCGGGTTTGGACTCGGCCTGTCCAGCGCCA | |
| | GCGCCGCCTTCATCAATGGCGCCCTTGGCAGCAA | |
| | GTGCAGACCTCAAAACGCGACGTCGTCGAAACTG | |
| | GACATCTACATAGAGGACATCCGGAGAGGGAAGC | |
| | ACGGGAGCGACTCCGGCTCGTACGACGCCCAAGG | |
| | AAGGTTCGTTCCGGAGAAGTTCGAGGAGATATTCG | |
| | CCAGGCACGCGAGGACGGTCCCCGACGCCCTGA | |
| | CCTCGGACGAGATCGACCAGCTGCTCCAAGCGAA | |
| | CAGAGAGCCCGGGGACTACAGCGGCTGGGCTGG | |
| | CGCGGAAGCGGAGTGGAAGATCCTGTACAGTCTC | |
| | GGCAAGGACGGGGACGGCCTCCTCCGCAAGGAC | |
| | GTCGCGAGGAGCGTCTACGACGGGACACTGTTCC | |
| | ACCGGCTCGCGCCCAGATGGAAATCTCCCGACAG | |
| | CGACATGGAGAGAAGCTGATAAGCGTGGTCCGGG | |
| | AGAACTGAACCGAGAGGACCGTCCTATTGATGTCG | |
| | TCTTGCGCTGGGCTGCTCTGAACTGAACAAGTCTG | |
| | GACATGCCGTCAAGCGACATGTGGGTGTGAACAC | |
| | TCTTTCGGGTCAGATTATTAACAAGAAGGGTGTGA | |
| | CCGTGTGAGTGCAAAAAAAAAAAAAA AA | |
| SEQ. ID NO: 13 | MAGEAEALATTAPLAPVTSQRKVRNDLEETLPKPYM | Arabidopsis thaliana |
| | ARALAAPDTEHPNGTEGHDSKGMSVMQQHVAFFDQ | Caleosin N-terminal |
| | NDDGIVYPWETYKGFRDLGFN | domain |
| SEQ. ID NO: 14 | MATHVLAAAAERNAALAPDAPLAPVTMERPVRTDLE | Sesamum indicum |
| | TSIPKPYMARGLVAPDMDHPNGTPGHVHDNLSVLQQ | Caleosin N-terminal |
| | HCAFFDQDDNGIIYPWETYSGLRQIGFN | domain |
| SEQ. ID NO: 15 | MAEEAASKAAPTDALSSVAAEAPVTRERPVRADLEV | Oryza sativa |
| | QIPKPYLARALVAPDVYHPEGTEGRDHRQMSVLQQH | Caleosin |
| | VAFFDLDGDGIVYPWETYGGLRELGFN | N-terminal domain |
| SEQ. ID NO: 16 | MAAEMERESLITEAPNAPVTAQRRVRNDLENSLPKP | Glycine max |
| | YLPRALKAPDTGHPNGTAGHRHHNLSVLQQHCAFFD | Caleosin N-terminal |
| | QDDNGIIYPWETYMGLRSIGFN | domain |
| SEQ. ID NO: 17 | ATGGCAGGAGAGGCAGAGGCTTTGGCCACGACGG | Arabidopsis thaliana |
| | CACCGTTAGCTCCGGTCACCAGTCAGCGAAAAGTA | Caleosin N-terminal |
| | CGGAACGATTTGGAGGAAACATTACCAAAACCATA | domain |
| | CATGGCAAGAGCATTAGCAGCTCCAGATACAGAGC | |
| | ATCCGAATGGAACAGAAGGTCACGATAGCAAAGGA | |
| | ATGAGTGTTATGCAACAACATGTTGCTTTCTTCGAC | |
| | CAAAACGACGATGGAATCGTCTATCCTTGGGAGAC | |
| | TTATAAGGGATTTCGTGACCTTGGTTTCAAC | |
| SEQ. ID NO: 18 | ATGGCAACTCATGTTTTGGCTGCTGCGGCGGAGA | Sesamum indicum |
| | GAAATGCTGCGTTGGCGCCGGACGCCCCGCTTGC | Caleosin N-terminal |
| | TCCGGTGACTATGGAGCGCCCAGTGCGCACTGAC | domain |
| | TTGGAGACTTCGATCCCGAAGCCCTATATGGCAAG | |
| | AGGATTGGTTGCACCTGATATGGATCACCCCAACG | |
| | GAACACCAGGCCATGTGCATGATAATTTGAGTGTG | |
| | CTGCAACAGCATTGTGCTTTCTTTGATCAGGATGAT | |
| | AACGGAATCATCTATCCATGGGAGACTTACTCTGG | |
| | ACTTCGCCAAATTGGTTTCAAT | |
| SEQ. ID NO: 19 | ATGGCGGAGGAGGCGGCTAGCAAGGCAGCGCCG | Oryza sativa |
| | ACCGATGCGCTGTCGTCCGTGGCGGCGGAGGCG | Caleosin N-terminal |
| | CCGGTGACGAGAGAACGGCCGGTCCGAGCGGAC | domain |
| | or | |
| | TTGGAAGTGCAGATTCCGAAGCCCTATTTGGCCCG | |
| | AGCTCTGGTTGCTCCGGACGTGTACCATCCTGAAG | |
| | GAACCGAGGGGCGTGACCACCGGCAGATGAGTGT | |
| | GCTGCAGCAGCATGTGGCTTTCTTCGACCTGGATG | |
| | GCGACGGTATCGTTTATCCATGGGAAACTTATGGA | |
| | GGACTACGGGAATTGGGCTTCAAC | |
| SEQ. ID NO: 20 | ATGGCTGCAGAGATGGAGAGGGAGTCATTGATAA | Glycine max |
| | CTGAAGCTCCTAATGCACCAGTTACTGCACAGAGA | Caleosin N-terminal |
| | AGGGTCAGAAATGACTTAGAAAATTCTCTACCAAAA | domain |
| | CCATACTTGCCAAGAGCATTGAAAGCTCCTGATAC | |
| | GGGTCACCCAAATGGAACAGCAGGCCACAGGCAC | |
| | CACAACTTATCTGTTCTTCAGCAGCATTGTGCTTTT | |
| | TTTGATCAAGATGACAATGGAATCATTTACCCTTGG | |
| | GAAACTTACATGGGGCTGCGTTCTATTGGATTTAAT | |
| SEQ. ID NO: 21 | PISSIFWTLLINLAFSYVTLPSWVPSPLLPVYIDNI | Arabidopsis thaliana |
| | | Caleosin Central |
| | | domain |
| SEQ. ID NO: 22 | VIASLIMAIVINVALSYPTLPGWIPSPFFPIYLYNI | Sesamum indicum |
| | | Caleosin Central |
| | | domain |
| SEQ. ID NO: 23 | VIVSFFLAIAINVGLSYPTLPSWIPSLLFPIHIKNI | Oryza sativa |
| | | Caleoson Central |
| | | domain |
| SEQ. ID NO: 24 | VVASVIMAIVINVGLSYPTLPNWFPSLLFPIYIHNI | Glycine max |
| | | Calesosin Central |
| | | domain |
| SEQ. ID NO: 25 | CCAATTTCCTCTATCTTTTGGACCTTACTCATAAAC | Arabidopsis thaliana |
| | TTAGCGTTCAGCTACGTTACACTTCCGAGTTGGGT | Caleosin Central |
| | GCCATCACCATTATTGCCGGTTTATATCGACAACAT | domain |
| | A | |
| SEQ. ID NO: 26 | GTGATAGCTTCCCTTATAATGGCTATCGTCATTAAT | Sesamum indicum |
| | GTGGCGCTGAGTTATCCTACTCTCCCGGGTTGGAT | Caleosin Central |
| | TCCTTCTCCTTTTTTCCCCATATATTTGTACAACATA | domain |
| SEQ. ID NO: 27 | GTGATTGTTTCGTTCTTTTTGGCGATAGCCATAAAC | Oryza sativa |
| | GTTGGTCTAAGCTACCCAACTCTGCCAAGCTGGAT | Caleosin Central |
| | ACCATCTCTCCTGTTCCCTATACACATAAAAAACAT | domain |
| | C | |
| SEQ. ID NO: 28 | GTTGTTGCATCTGTTATTATGGCTATTGTTATCAAT | Glycine max |
| | GTTGGATTGAGTTACCCCACTCTACCTAATTGGTTC | Caleosin Central |
| | CCTTCTCTCCTTTTTCCTATCTACATACACAACATA | domain |
| SEQ. ID NO: 29 | HKAKHGSDSSTYDTEGRLSNKVEWILLYILAKDEDGF | Arabidopsis thaliana |
| | LSKEAVRGCFDGSLFEQIAKERANSRKQD | Caleosin C-terminal |
| | | domain |
| SEQ. ID NO: 30 | HKAKHGSDSGTYDTEGRYLPMNFENLFSKHARTMP | Sesamum indicum |
| | DRLTLGELWSMTEANREAFDIFGWIASKMEWTLLYIL | Caleosin C-terminal |
| | ARDQDGFLSKEAIRRCYDGSLFEYCAKMQRGAEDK | domain |
| | MK | |
| SEQ. ID NO: 31 | HRAKHGSDSSTYDNEGRFMPVNFESIFSKNARTAPD | Oryza sativa |
| | KLTFGDIWRMTEGQRVALDLLGRIASKGEWILLYVLA | Caleosin C-terminal |
| | KDEEGFLRKEAVRRCFDGSLFESIAQQRREAHEKQK | domain |
| SEQ. ID NO: 32 | HKAKHGSDSGVYDTEGRYVPANIENIFSKYARTVPDK | Glycine max |
| | LTLGELWDLTEGNRNAFDIFGWLAAKFEWGVLYILAR | Caleosin C-terminal |
| | DEEGFLSKEAVRRCFDGSLFEYCAKMHTTSDAKMS | domain |
| SEQ. ID NO: 33 | CACAAAGCCAAGCATGGGAGTGATTCGAGCACCTA | Arabidopsis thaliana |
| | TGACACCGAAGGAAGGCTTTCAAACAAAGTTGAAT | Caleosin C-terminal |
| | GGATACTACTCTATATTCTTGCTAAGGACGAAGAT | domain |
| | GGTTTCCTATCTAAAGAAGCTGTGAGAGGTTGCTT | |
| | TGATGGAAGTTTATTTGAACAAATTGCCAAAGAGA | |
| | GGGCCAATTCTCGCAAACAAGAC | |
| SEQ. ID NO: 34 | CACAAGGCCAAACATGGAAGCGACTCCGGAACCT | Sesamum indicum |
| | ATGATACTGAAGGAAGGTACCTACCTATGAATTTTG | Caleosin C-terminal |
| | AGAACCTGTTCAGCAAGCATGCCCGGACAATGCC | domain |
| | CGATAGGCTCACTCTAGGGGAGCTATGGAGCATG | |
| | ACTGAAGCTAACAGAGAAGCATTTGACATTTTCGG | |
| | CTGGATCGCAAGCAAAATGGAGTGGACTCTCCTCT | |
| | ACATTCTTGCAAGAGACCAGGACGGTTTCCTGTCG | |
| | AAAGAAGCCATCAGGCGGTGTTACGATGGCAGTTT | |
| | GTTCGAGTACTGTGCAAAGATGCAAAGGGGAGCC | |
| | GAGGACAAGATGAAA | |
| SEQ. ID NO: 35 | CACAGGGCTAAGCACGGCAGCGATAGCTCGACGT | Oryza sativa |
| | ACGACAACGAGGGAAGGTTTATGCCGGTCAATTTC | C-terminal domain |
| | GAGAGCATCTTCAGCAAGAACGCCCGCACGGCGC | |
| | CGGACAAGCTCACGTTCGGCGATATCTGGCGGAT | |
| | GACCGAAGGCCAAAGGGTGGCGCTCGACTTGCTT | |
| | GGGAGGATCGCGAGTAAGGGGGAGTGGATATTGC | |
| | TCTACGTGCTTGCGAAAGATGAGGAAGGATTCCTC | |
| | AGGAAGGAGGCTGTTCGCCGCTGCTTCGATGGGA | |
| | GCCTATTCGAGTCGATTGCCCAGCAGAGAAGGGA | |
| | GGCACATGAGAAGCAGAAG | |
| SEQ. ID NO: 36 | CACAAAGCAAAGCATGGGAGTGACTCTGGAGTTTA | Glycine max |
| | TGACACAGAAGGACGTTATGTGCCAGCAAATATTG | Caleosin C-terminal |
| | AGAACATATTCAGTAAGTATGCTCGTACAGTACCT | domain |
| | GACAAGCTCACACTTGGGGAGCTCTGGGACTTGA | |
| | CAGAGGGAAACCGAAATGCTTTTGACATATTTGGC | |
| | TGGCTTGCAGCAAAATTTGAATGGGGGGTTCTGTA | |
| | CATTCTGGCAAGGGATGAGGAAGGTTTCCTGTCTA | |
| | AAGAAGCTGTTAGAAGATGCTTTGATGGGAGCTTA | |
| | TTTGAATACTGTGCTAAAATGCATACTACTAGTGAT | |
| | GCCAAGATGAGT | |
| SEQ. ID NO: 37 | ENLYFQS | Synthetic Linker |
| SEQ. ID. NO: 38 | GAGAACCTCTACTTCCAATCG | Synthetic linker |
| SEQ. ID NO: 39 | CITGDALVALPEGESVRIADIVPGARPNSDNAIDLKVL | Synthetic Linker |
| | DRHGNPVLADRLFHSGEHPVYTVRTVEGLRVTGTAN | (intein) |
| | HPLLCLVDVAGVPTLLWKLIDEIKPGDYAVIQRSAFSV | |
| | DCAGFARGKPEFAPTTYTVGVPGLVRFLEAHHRDPD | |
| | AQAIADELTDGRFYYAKVASVTDAGVQPVYSLRVDT | |
| | ADHAFITNGFVSHA | |
| SEQ. ID. NO: 40 | TGCATCACGGGAGATGCACTAGTTGCCCTACCCGA | Synthetic linker |
| | GGGCGAGTCGGTACGCATCGCCGACATCGTGCCG | (Intein) |
| | GGTGCGCGGCCCAACAGTGACAACGCCATCGACC | |
| | TGAAAGTCCTTGACCGGCATGGCAATCCCGTGCTC | |
| | GCCGACCGGCTGTTCCACTCCGGCGAGCATCCGG | |
| | TGTACACGGTGCGTACGGTCGAAGGTCTGCGTGT | |
| | GACGGGCACCGCGAACCACCCGTTGTTGTGTTTG | |
| | GTCGACGTCGCCGGGGTGCCGACCCTGCTGTGGA | |
| | AGCTGATCGACGAAATCAAGCCGGGCGATTACGC | |
| | GGTGATTCAACGCAGCGCATTCAGCGTCGACTGT | |
| | GCAGGTTTTGCCCGCGGGAAACCCGAATTTGCGC | |
| | CCACAACCTACACAGTCGGCGTCCCTGGACTGGT | |
| | GCGTTTCTTGGAAGCACACCACCGAGACCCGGAC | |
| | GCCCAAGCTATCGCCGACGAGCTGACCGACGGGC | |
| | GGTTCTACTACGCGAAAGTCGCCAGTGTCACCGAC | |
| | GCCGGCGTGCAGCCGGTGTATAGCCTTCGTGTCG | |
| | ACACGGCAGACCACGCGTTTATCACGAACGGGTT | |
| | CGTCAGCCACGCT | |
| SEQ. ID NO: 41 | TCGCTGAGGCTTGACATGATTGGTGCGTATGTTTG | Synthetic-promoter |
| | TATGAAGCTACAGGACTGATTTGGCGGGCTATGAG | Hsp70A-Rbc52 |
| | GGCGGGGGAAGCTCTGGAAGGGCCGCGATGGGG | |
| | CGCGCGGCGTCCAGAAGGCGCCATACGGCCCGC | |
| | TGGCGGCACCCATCCGGTATAAAAGCCCGCGACC | |
| | CCGAACGGTGACCTCCACTTTCAGCGACAAACGA | |
| | GCACTTATACATACGCGACTATTCTGCCGCTATAC | |
| | ATAACCACTCAGCTAGCTTAAGATCCCATCAAGCTT | |
| | GCATGCCGGGCGCGCCAGAAGGAGCGCAGCCAA | |
| | ACCAGGATGATGTTTGATGGGGTATTTGAGCACTT | |
| | GCAACCCTTATCCGGAAGCCCCCTGGCCCACAAA | |
| | GGCTAGGCGCCAATGCAAGCAGTTCGCATGCAGC | |
| | CCCTGGAGCGGTGCCCTCCTGATAAACCGGCCAG | |
| | GGGGCCTATGTTCTTTACTTTTTTACAAGAGAAGTC | |
| | ACTCAACATCTTAAA | |
| SEQ. ID NO: 42 | PSWVPSPLLP | Arabidopsis thaliana |
| | | Caleosin proline knot |
| SEQ. ID NO: 43 | PGWIPSPFFP | Sesamum indicum |
| | | Caleosin proline knot |
| SEQ. ID NO: 44 | PSWIPSLLFP | Oryza sativa |
| | | Caleosin proline knot |
| SEQ. ID NO: 45 | PNWFPSLLFP | Glycine max |
| | | Caleosin proline knot |
| SEQ. ID NO: 46 | CCGAGTTGGGTGCCATCACCATTATTGCCG | Arabidopsis thaliana |
| | | Caleosin proline knot |
| SEQ. ID NO: 47 | CCGGGTTGGATTCCTTCTCCTTTTTTCCCCATATAT | Sesamum indicum |
| | TTGTACAACATA | |
| SEQ. ID NO: 48 | CCAAGCTGGATACCATCTCTCCTGTTCCCT | Oryza sativa |
| | | Caleosin proline knot |
| SEQ. ID NO: 49 | CCTAATTGGTTCCCTTCTCTCCTTTTTCCT | Glycine max |
| | | Caleosin proline knot |
| SEQ. ID NO: 50 | MQQHVAFFDQNDDGIVYPWETYKGFRDL | Arabidopsis thaliana |
| | | Caleosin Ca binding |
| | | domain |
| SEQ. ID NO: 51 | LQQHCAFFDQDDNGIIYPWETYSGLRQI | Sesamum indicum |
| | | Caleosin Ca binding |
| | | domain |
| SEQ. ID NO: 52 | PDVYHPEGTEGRDHRQMSVLQQHVAFFDLDGDGIV | Oryza sativa |
| | YPWETYGGLRELGFN | Caleosin Ca Binding |
| | | domain |
| SEQ. ID NO: 53 | PDTGHPNGTAGHRHHNLSVLQQHCAFFDQDDNGIIY | Glycine max |
| | PWETYMGLRSIGFN | Caleosin Ca binding |
| | | domain |
| SEQ. ID NO: 54 | ATGCAACAACATGTTGCTTTCTTCGACCAAAACGA | Arabidopsis thaliana |
| | CGATGGAATCGTCTATCCTTGGGAGACTTATAAGG | Calesosin Ca binding |
| | GATTTCGTGACCTT | domain |
| SEQ. ID NO: 55 | CTGCAACAGCATTGTGCTTTCTTTGATCAGGATGAT | Sesamum indicum |
| | AACGGAATCATCTATCCATGGGAGACTTACTCTGG | Caleosin Ca binding |
| | ACTTCGCCAAATT | domain |
| SEQ. ID NO: 56 | CCGGACGTGTACCATCCTGAAGGAACCGAGGGGC | Oryza sativa |
| | GTGACCACCGGCAGATGAGTGTGCTGCAGCAGCA | Caleosin Ca binding |
| | TGTGGCTTTCTTCGACCTGGATGGCGACGGTATCG | domain |
| | TTTATCCATGGGAAACTTATGGAGGACTACGGGAA | |
| | TTGGGCTTCAAC | |
| SEQ. ID NO: 57 | CCTGATACGGGTCACCCAAATGGAACAGCAGGCC | Glycine max |
| | ACAGGCACCACAACTTATCTGTTCTTCAGCAGCAT | Caleosin Ca binding |
| | TGTGCTTTTTTTGATCAAGATGACAATGGAATCATT | domain |
| | TACCCTTGGGAAACTTACATGGGGCTGCGTTCTAT | |
| | TGGATTTAAT | |

Although the invention has been described with reference to certain specific embodiments, various modifications thereof will be apparent to those skilled in the art without departing from the spirit and scope of the invention. All such modifications as would be apparent to one skilled in the art are intended to be included within the scope of the following claims.

1. A method of producing an algal chloroplast enriched for recombinant polypeptide, the method comprising:
subjecting growing algal cells comprising a recombinant polypeptide to non-homeostatic conditions to target the recombinant polypeptide to the algal chloroplast; wherein the recombinant polypeptide is a fusion polypeptide comprising an oil body protein or fragment thereof, wherein the oil body protein is a caleosin or a fragment thereof.

2. (canceled)

3. The method according to claim 1, wherein the caleosin is a protein encoded by a nucleic acid sequence having the sequence set forth in SEQ.ID NO: 7 to SEQ.ID NO: 12 or wherein the caleosin has an amino acid sequence as set forth in any one of SEQ. ID NO: 1 to 6 or a fragment thereof.

4. (canceled)

5. The method of claim 1, comprising the step of introducing a nucleic acid encoding the recombinant polypeptide into the algal cell.

6. The method of claim 5, wherein the nucleic acid encoding the fusion polypeptide comprises one or more algal cell control elements.

7. The method of claim 1, comprising isolating the algal chloroplasts.

8. Chloroplasts produced by the method of claim 7.

9. A method of producing algal chloroplasts enriched for recombinant polypeptide, the method comprising:
(a) introducing a nucleic acid into algal cells, the nucleic acid comprising as operably linked components
(i) a nucleic acid encoding a fusion polypeptide comprising an oil body protein or fragment thereof to provide targeting to the algal chloroplast and a polypeptide of interest; and
(ii) a nucleic acid sequence capable of controlling expression in an algal cell;
(b) subjecting the algal cells in a growth medium to non-homeostatic conditions to target the fusion polypeptide to the algal chloroplast; and
(c) optionally isolating the algal chloroplasts, wherein the oil body protein is a caleosin or a fragment thereof.

10. (canceled)

11. The method according to claim 9, wherein the caleosin is a protein encoded by a nucleic acid sequence having the sequence set forth in SEQ.ID NO: 7 to SEQ.ID NO: 12 or wherein the caleosin has an amino acid sequence as set forth in any one of SEQ. ID NO: 1 to 6 or a fragment thereof.

12. (canceled)

13. Chloroplasts produced by the method of claim 9.

14. The method according to claim 1, wherein the non-homeostatic conditions comprise limiting one or more nutrients in the growth medium such that after a period of growth the amount of the one or more nutrients is insufficient for homeostatic algal cell growth, wherein in the one or more nutrients are optionally nitrogen, phosphorus or combination thereof.

15. The method according to claim 1, wherein the non-homeostatic conditions are an exogenous stress factor, wherein the exogenous stress factor is selected from the group consisting of a non-homeostatic pH, a non-homeostatic salinity, and a non-homeostatic light intensity.

16-19. (canceled)

20. A method of producing a recombinant protein comprising the method of claim 9 and further comprising isolating the recombinant polypeptide from the chloroplast.

21. The method according to claim 1, wherein the algal cell is selected from the group of algal cells consisting of cyanobacteria (Cyanophyceae), green algae (Chlorophyceae), diatoms (Bacillariophyceae), yellow-green algae (Xanthophyceae), golden algae (Chrysophyceae), red algae (Rhodophyceae), brown algae (Phaeophyceae), dinoflagellates (Dinophyceae) and pico-plankton (Prasinophyceae and Eustigmatophyceae).

22. The method according to claim 9, a wherein the algal call is an algal cell belonging to the genus Clamydomonas, or Chlorella.

23-50. (canceled)
